(19) United States
(12) Patent Application Publication
(10) Pub. No.: US 2006/0051436 A1
(43) Pub. Date: Mar. 9, 2006

(54) Composition for controlling excessive calorie intake-induced disorder, food product for controlling excessive calorie intake-induced disorder, skin preparation for external use for controlling excessive calorie intake-induced disorder, and controlling agent for excessive calorie intake-induced disorder

(75) Inventors: Hiroshi Ogawa, Tondabayashi-shi (JP); Toshio Mitsunaga, Kyoto-shi (JP); Toshinori Kamisako, Sakai-shi (JP); Yukio Kawamura, Nara-shi (JP)

Correspondence Address: DARBY & DARBY P.C., P. O. BOX 5257, NEW YORK, NY, 10150-5257, US

(73) Assignee: Towa Corporation Kabushiki Kaisha, Tokyo (JP)

(21) Appl. No.: 11/219,354

(22) Filed: Sep. 2, 2005

Related U.S. Application Data
(63) Continuation-in-part of application No. 11/031,732, filed on Jan. 6, 2005.

(30) Foreign Application Priority Data
Apr. 7, 2004 (JP) 2004-113027

(51) Int. Cl. A61K 36/185; A61K 31/44

(52) U.S. Cl. 424/725; 514/356

(57) ABSTRACT

A composition contains 0.1 to 10 g of dried Hercampuri extract obtained by extracting the essence of dried whole Hercampuri plant with 60 to 80% aqueous ethanol. It has a function of prevention or curative treatment of lifestyle-related disorders induced by accumulation of visceral fat resulting from excessive calorie intake. It is useful for maintaining, and improving health.

### INCORPORATION BY REFERENCE

The present application claims priority under 35 U.S.C. §119 to Japanese Patent Application No. 2004-113027 filed on Apr. 7, 2004. The content of the application is incorporated herein by reference in its entirety.

### TECHNICAL FIELD OF THE INVENTION

The present invention relates to a composition, a food product, a skin preparation for external use, and a controlling agent, each of which contains, at least, Hercampuri and has a function of controlling disorders induced by excessive calorie intake.

### BACKGROUND OF THE INVENTION

In recent years, Japanese dietary habits have exhibited an increase in the consumption of excessive fat or calories as a result of changes in lifestyles, including the Westernization of dietary habits. This has resulted in a sharp increase in the morbidity of lifestyle-related diseases, such as hyperlipidemia, obesity, hypertension, and diabetes. Various remedies are used to treat hypertension, high cholesterol, and diabetes, all of which are typical examples of lifestyle-related diseases. As described in, for example, Japanese Patent Laid-open Nos. 2002-363095, 06-345664, and 06-135839, effectiveness of these respective remedies are accepted to a certain level.

However, these therapeutics target sick people, in other words those who have an disorder, and have not inconsiderable adverse effects, resulting in significant resistance regarding long-term usage. Therefore, there is a great demand for development of a functional food product that is a safe, non-medicinal agent with superior effectiveness in prevention and curative treatment of lifestyle-related diseases and intended for people who are borderline patients for such diseases. However, no product has ever been developed to satisfy this demand completely. At present there is a great demand for development of a composition that is effective in the prevention and curative treatment of lifestyle-related diseases, and in particular, that has curative properties against diabetes.

Among the lifestyle-related diseases mentioned above, diabetes is particularly a problem in that the number of diabetics, as well as incipient diabetics, has been on a sharp increase over recent years. To be more specific, 2002 Fact-finding Survey Report (bulletin) on Diabetes reported that there were approximately 7.4 million who “had a strong possibility of being diabetic” and approximately 16.2 million “for whom diabetes could not be ruled out”. These numbers have been clearly increasing every year. Of the types of diabetes, the increase in Type 2 diabetes, which is associated with obesity, insulin resistance, hyperlipidemia, or hypertension, is particularly conspicuous.

Hercampuri is a perennial dicotyledon growing in the Andean highlands in Peru. This plant has been used from generation to generation from Pre-Inca times, as an effective cure-all for suppressing or alleviating stomach colic, chronic gastritis, and liver functions disorders. It is known that Hercampuri has been effective in revitalizing kidney/liver functions, treating hepatitis, lowering malaria fever, purifying blood, and stimulating bile secretions, as well as antidiabetic effect. Hercampuri is typically taken in the form of a health tea, which is made by brewing whole dried Hercampuri plants.

Xanthone derivatives are known to be among the principal components of Hercampuri, among which Bellidifolin is found in the largest amounts.

As is described, for example, by P. Basnet and three other embers in P.507-511 of Planta Medica (Germany), 1994, Vol. 60 (“Bellidifolin obtained from Swertia japonica is highly effective in reducing blood sugar levels in rats with streptozotocin (STZ)-induced diabetes”) and by P. Basnet and four other members in P.401-405 of Planta Medica (Germany), 1995, Vol. 61 (“Bellidifolin is effective in reducing blood sugar levels by enhancing intake of glucose into fibroblast in rats with streptozotocin (STZ)-induced diabetes”), it is widely known that Bellidifolin has a function of reducing blood sugar levels in streptozotocin (STZ)-induced diabetic rats.

Mangiferin is another xanthone derivative contained in Hercampuri. As is described, for example, by H. Ichiki and six other members in P.1389-1390 of Biological Pharmaceutical Bulletin (Japan), 1998, Vol. 21 (“Mangiferin is a novel antidiabetic compound”), it is known that Mangiferin has a function of reducing blood sugar levels and increasing insulin sensitivity in KK-Ay mice, which are regarded as a model of Type 2 diabetes.

As is described in, for example, Japanese Patent Laid-open No. 2004-172, it has recently been reported that Hercampuri is effective in preventing or alleviating lifestyle-related diseases, by performing such functions as suppressing the increase in blood pressure, suppressing LDL cholesterol, increasing HDL cholesterol, or treating diabetes or obesity.

However, the effects of Hercampuri have not yet been clearly proven nor have physiological functions of the components of Hercampuri been clarified. Moreover, neither the aforementioned functions of Hercampuri against diabetes nor concrete antiobesity effect of Hercampuri have been made clear.

In view of the problems described above, an object of the invention is to provide a composition, a food product, a skin preparation for external use, and a controlling agent, each of which contains, at least, Hercampuri and has a function of controlling disorders induced by excessive calorie intake.

### SUMMARY OF THE INVENTION

A composition for controlling disorders induced by excessive calorie intake according to the present invention contains, at least, Hercampuri and has a function of controlling an interlinked complex of diseases induced by excessive calorie intake.

As the aforementioned composition contains, at least, Hercampuri, the composition possesses significant potential for the capability of controlling an interlinked complex of diseases induced by excessive calorie intake.

A composition for controlling disorders induced by excessive calorie intake according to the present invention has a function of controlling lifestyle-related disorders induced by accumulation of visceral fat.

As the aforementioned composition contains, at least, Hercampuri, the composition possesses significant potential for the capability of controlling lifestyle-related disorders induced by accumulation of visceral fat.

A composition for controlling disorders induced by excessive calorie intake according to the present invention has a function of controlling diabetes.

As the aforementioned composition contains, at least, Hercampuri, the composition possesses significant potential for the capability of controlling diabetes.

A composition for controlling disorders induced by excessive calorie intake according to the present invention has a function of controlling Type 2 diabetes.

As the aforementioned composition contains, at least, Hercampuri, the composition possesses significant potential for the capability of controlling Type 2 diabetes.

A composition for controlling disorders induced by excessive calorie intake according to the present invention has a function of improving serum lipid conditions and liver lipid conditions.

As the aforementioned composition contains, at least, Hercampuri, the composition possesses significant potential for the capability of improving serum lipid conditions and liver lipid conditions.

A composition for controlling disorders induced by excessive calorie intake according to the present invention has a function of lowering serum glucose levels.

As the aforementioned composition contains, at least, Hercampuri, the composition possesses significant potential for the capability of lowering serum glucose levels.

A composition for controlling disorders induced by excessive calorie intake according to the present invention has a function of improving free fatty acid levels.

As the aforementioned composition contains, at least, Hercampuri, the composition possesses significant potential for the capability of improving free fatty acid levels.

A composition for controlling disorders induced by excessive calorie intake according to the present invention has a function of reducing insulin resistance.

As the aforementioned composition contains, at least, Hercampuri, the composition possesses significant potential for the capability of reducing insulin resistance.

A composition for controlling disorders induced by excessive calorie intake according to the present invention has a function of decreasing cholesterol levels.

As the aforementioned composition contains, at least, Hercampuri, the composition possesses significant potential for the capability of decreasing cholesterol levels.

A composition for controlling disorders induced by excessive calorie intake according to the present invention has a function of reducing fat.

As the aforementioned composition contains, at least, Hercampuri, the composition possesses significant potential for the capability of reducing fat.

A composition for controlling disorders induced by excessive calorie intake according to the present invention has a function of reducing visceral fat.

As the aforementioned composition contains, at least, Hercampuri, the composition possesses significant potential for the capability of reducing visceral fat.

A composition for controlling disorders induced by excessive calorie intake according to the present invention has a function of reducing subcutaneous fat.

As the aforementioned composition contains, at least, Hercampuri, the composition possesses significant potential for the capability of reducing subcutaneous fat.

A composition for controlling disorders induced by excessive calorie intake according to the present invention has preventing and curative properties effective against risk factors for metabolic syndrome.

As the aforementioned composition contains, at least, Hercampuri, the composition possesses significant potential for preventing or alleviating risk factors for metabolic syndrome.

A composition for controlling disorders induced by excessive calorie intake according to the present invention contains, at least, alcohol-extracted Hercampuri.

As the aforementioned composition contains, at least, alcohol-extracted Hercampuri, the composition is more easily ingested.

A composition for controlling disorders induced by excessive calorie intake according to the present invention contains, at least, Hercampuri extracted with aqueous alcohol containing 60 to 80% ethanol.

As the aforementioned composition contains, at least, Hercampuri extracted with aqueous alcohol containing 60 to 80% ethanol, the composition is more easily ingested.

A food product for controlling disorders induced by excessive calorie intake according to the present invention contains a composition for controlling disorders induced by excessive calorie intake.

Eating the food containing a composition for controlling disorders induced by excessive calorie intake presents the possibility of controlling an interlinked complex of diseases induced by excessive calorie intake.

A skin preparation for external use for controlling disorders induced by excessive calorie intake according to the present invention contains a composition for controlling disorders induced by excessive calorie intake.

Using the skin preparation containing a composition for controlling disorders induced by excessive calorie intake presents the possibility of controlling an interlinked complex of diseases induced by excessive calorie intake.

A controlling agent for controlling disorders induced by excessive calorie intake according to the present invention contains a composition for controlling disorders induced by excessive calorie intake.

Using the skin preparation containing a composition for controlling disorders induced by excessive calorie intake presents the possibility of controlling an interlinked complex of diseases induced by excessive calorie intake.

### BRIEF DESCRIPTION OF THE DRAWINGS

FIG. 1 is a graph showing the inhibitory activity on the α-amylase (1 unit/ml) by Hercampuri extract (2 mg substrate/ml) that contains Hercampuri.

FIG. 2 is a graph showing the inhibitory activity on the α-amylase (1 unit/ml) by Mangiferin (2 mg substrate/ml).

FIG. 3 is a graph showing the inhibitory activity on the α-amylase (1 unit/ml) by the aforementioned Hercampuri (5 mg substrate/ml).

FIG. 4 is a graph showing the inhibitory activity on the α-amylase (1 unit/ml) by Mangiferin (5 mg substrate/ml).

FIG. 5 is a graph showing the inhibitory activity on the α-amylase (0.5 unit/ml) by the aforementioned Hercampuri (5 mg substrate/ml).

FIG. 6 is a graph showing the inhibitory activity on the α-amylase (0.5 unit/ml) by Mangiferin (5 mg substrate/ml).

FIG. 7 is a graph showing concentrations of the aforementioned Hercampuri extract when inhibiting enzyme activity by 50%.

FIG. 8 is a graph showing the correlation between the inhibitory activities of the aforementioned Hercampuri extract and Mangiferin.

FIG. 9 is a graph showing the inhibitory activity on the α-glucosidase by Mangiferin (25 mg substrate/ml).

FIG. 10 is a graph showing distribution of cholesterol, phospholipid, and triglyceride in whole liver of each group of rats.

FIG. 11 is a graph showing change in body weight of each group of rats.

FIG. 12 is a graph showing change in food intake of each group of rats.

FIG. 13 is a graph showing distribution of cholesterol of each group of rats.

FIG. 14 is a graph showing phospholipid distribution of each group of rats.

FIG. 15 is a graph showing triglyceride distribution of each group of rats.

FIG. 16 is a graph showing the influence exerted by the ingestion of tablets produced from Hercampuri extract powder on serum triglyceride level.

FIG. 17 is a graph showing the influence exerted by the ingestion of tablets produced from Hercampuri extract powder on total cholesterol level in serum.

FIG. 18 is a multislice CT image of the abdomen at the height of the navel of subject A taken when subject A commenced taking the aforementioned tablets produced from Hercampuri extract powder.

FIG. 19 is a multislice CT image of the abdomen at the height of the navel of subject A taken after twelve weeks of the ingestion of the tablets produced from Hercampuri extract powder.

FIG. 20 is a multislice CT image of the abdomen at the height of the navel of subject B taken when subject A commenced taking the aforementioned tablets produced from Hercampuri extract powder.

FIG. 21 is a multislice CT image of the abdomen at the height of the navel of subject B taken after twelve weeks of the ingestion of the tablets produced from Hercampuri extract powder.

FIG. 22 is a schematic illustration to explain lifestyle-related diseases caused by accumulation of visceral fat.

FIG. 23 is a schematic illustration to explain sugar/lipid metabolism disorder caused by accumulation of visceral fat.

### DETAILED DESCRIPTION OF THE INVENTION

Next, a functional food product that contains Hercampuri and is in accordance with a first embodiment of the present invention is explained hereunder. First, dry Hercampuri, i.e. dried whole Hercampuri plant (roots, stems, and leaves), is ground to a particle size of approximately 30 to 100 mesh. The Hercampuri is also called Hercampure. The ground dry Hercampuri is then immersed in an aqueous alcohol for 48 to 72 hours at room temperature (approximately 40° C. when a higher extraction efficiency is desired) in order to obtain an extract of Hercampuri.

The liquid extract may directly undergo concentration solidifying under reduced pressure or may be spray-dried by means of adding a drying agent, such as dextrin or sorbitol, to the liquid and spraying the mixture. It is also possible to dissolve the dried extract in water and treat the solution with activated carbon, ion-exchange-resin processing, or the like to refine the extract further. The alcohol used is low alcohol, such as ethanol or propanol. It is desirable that the alcohol is an aqueous alcohol with normal contents of approximately 60 to 80%. As a result of the process described above, alcohol-extracted dried Hercampuri extract is obtained.

When provided as a functional food product for preventing or alleviating lifestyle-related diseases, by performing such functions as suppressing the increase in blood pressure, suppressing LDL cholesterol while increasing HDL cholesterol, or treating diabetes or obesity, the aforementioned dried Hercampuri extract extracted with the aqueous alcohol may be in any form selected from among powder, granule, tablet, sugar-coated tablet, capsule, liquid, and syrup. Whichever form is chosen, the product may be prepared with an auxiliary or a flavor additive. Examples of the excipient or the diluent that can be used include gelatin, various saccharides, starch, fatty acids, their salts, fats and oils, talc, physiological saline, and other masking agents.

Although the product formulated as above may be ingested as is, it may be conveniently ingested in various prepared foods, confectioneries, or candies. Although the appropriate dosage varies considerably, depending on individual differences, the normal daily dosage for an adult is in the range of 0.01 g to 10 g, more preferably 0.1 g to 10 g, of alcohol-extracted dried Hercampuri extract containing 8.5% moisture and 83% sugar.

The functional food product containing alcohol-extracted dried Hercampuri extract according to the present invention and which is effective in preventing or alleviating lifestyle-related diseases is explained in more detail hereunder, referring to conducted tests.

(Test 1)

In order to prepare Hercampuri extract powder, first of all, an aqueous alcohol was prepared by diluting 98% ethanol to 70% with water purified by means of activated carbon and ion-exchange resin. 120 kg of dried whole Hercampuri plant (roots, stems, and leaves; merchandise sold by TOWA Corporation) ground to a particle size of 60 mesh was immersed in 600 l of the diluted ethanol for 72 hours at room temperature.

Then, liquid extract was produced by filtering off the solid substances, and edible dextrin was mixed in at a relative ratio of 2% by mass into the liquid extract. Through spray drying of the mixture, 15 to 20 kg of the alcohol-extracted, spray-dried Hercampuri extract powder was obtained. The result of analysis of the Hercampuri extract powder is shown in Table 1.

TABLE 1

| | Analytic values |
|---|---|
| | [percent by mass] |
| Moisture | 8.5 |
| Protein | 1.5 |
| Lipid | 1.2 |
| Water-soluble dietary fiber | 1.3 |
| Water-insoluble dietary fiber | 3.0 |
| Sugar* | 83.0 |
| Ash | 1.5 |

*Fiber is not included in sugar

(Test 2)

Next, an animal diet containing 1% by mass of Hercampuri extract powder obtained through Test 1 was prepared. In order to serve as a control diet, casein feed containing no Hercampuri was prepared so that the casein feed had an AIN-93 composition (disclosed by Reeves et al. in Journal of Nutrition, P.1923, vol. 123, 1993). The respective compositions of the feed containing Hercampuri extract powder and the control diet are shown in Table 2. AIN-93 mentioned above is a standard purified diet composition disclosed in 1993 by U.S. American Institutes of Nutrition for research and study of nutrition using mice or rats.

TABLE 2
[percent by mass]

| | Control | Hercampuri-enriched |
|---|---|---|
| Ingredients | diet | diet |
| Hercampuri Ext. powder | — | 1.00 |
| Milk casein | 20.00 | 20.00 |
| L-Cystine | 0.30 | 0.30 |
| Soybean oil | 10.00 | 10.00 |
| Mineral mixture | 3.50 | 3.50 |
| (AIN-93G) | | |
| Vitamin mixture | 1.00 | 1.00 |
| (AIN-93) | | |
| Cellulose powder | 5.00 | 5.00 |
| Corn starch | 36.75 | 35.75 |
| α-Corn starch | 13.20 | 13.20 |
| Sucrose | 10.00 | 10.00 |
| Choline bitartrate | 0.25 | 0.25 |

An experiment was performed using stroke-prone spontaneously hypertensive rats (hereinafter referred to as SHRSP), which are known as a model animal prone to essential hypertension. To be more specific, SHRSP that were 16 to 17 weeks old (received from Animal Facilities for Experimental Medicine, Kinki University School of Medicine) were divided into groups, each of which consisted of six rats, and kept for six weeks at a temperature of 22.0±2.0° C. and humidity of 55.0±5.0% with 12-hour changeover of the ambient light (the light period being from 7:00 am to 7:00 pm and the dark period from 7:00 pm to 7:00 am). The rats had free access to feed and drinking water (tap water). The experiment was performed in accordance with Animal Experiment Guideline of Kinki University School of Medicine. During the experiment, the body weights and the amounts of food intake were measured every other day. A result of measurement, i.e. the averages of the body weights of the six rats of each group, is shown in Table 3.

TABLE 3
Change in body weight
(Average weight (g) of each group of 6 subjects

| Elapsed | | Hercampuri- |
|---|---|---|
| time | Control | enriched |
| (days) | diet | diet |
| 0 | 266 | 269 |
| 2 | 264 | 272 |
| 4 | 259 | 261 |
| 6 | 265 | 267 |
| 8 | 278 | 278 |
| 10 | 284 | 286 |
| 12 | 283 | 280 |
| 14 | 278 | 278 |
| 16 | 287 | 288 |
| 18 | 288 | 287 |
| 20 | 291 | 291 |
| 22 | 289 | 293 |
| 24 | 296 | 296 |
| 26 | 299 | 294 |
| 28 | 294 | 286 |
| 30 | 298 | 299 |
| 32 | 301 | 301 |
| 34 | 295 | 298 |
| 36 | 305 | 305 |
| 38 | 305 | 306 |
| 40 | 308 | 313 |
| 42 | 313 | 312 |

Once a week from the 3rd week of the experiment, the blood pressure of each rat was measured without anesthesia by the tail-cuff method using a non-invasive automatic sphygmomanometer (SOFTRON BP-98A: product of SOFTRON Co., Ltd., Tokyo). To be more specific, after preheating SHRSP in a heater for the period ranging from 4 to 5 minutes at a temperature of 38 to 40° C., the sphygmomanometer is attached to the rat to measure its blood pressure for 5 minutes at 38° C. without anesthesia. At that time, the systolic and diastolic tail arterial pressures were measured 5 times for each subject, and the averages of the measured values of the two types of blood pressures were respectively recorded as the maximum and minimal blood pressures. The change in maximum blood pressures is shown in Table 4.

TABLE 4

Maximum blood pressure

| | | Hercampuri-enriched |
|---|---|---|
| | Control diet | diet |
| 3rd weed | 245 ± 2 | 241 ± 6 |
| 4th week | 262 ± 3 | 254 ± 6 |
| 5th week | 264 ± 6 | 256 ± 9 |
| 6th week | 271 ± 3 | 258 ± 3** |

**p (significant difference from the control) < 0.01;
Mean ± Standard error of the mean

Six week after the initiation of the experiment, without prior abstinence from feed, each rat was incised at the abdomen under ether anesthesia to remove all of its blood via the abdominal aorta with an injection syringe. This operation was performed in the morning. The blood obtained by the operation was set aside for 1 hour at room temperature and subsequently at 4° C. for a period in the range of 1 to 2 hours. Thereafter, blood serum was separated by means of centrifugation at 2500 rpm at 4° C. for 10 minutes.

A commercially available UV kit (GOT-UV Test Wako; product of Wako Pure Chemical Industries, Ltd.) was used for measurement of glutamic oxaloacetic transaminase (GOT) activity. Another commercial UV kit (GPT-UV Test Wako; product of Wako Pure Chemical Industries, Ltd.) was used for measurement of glutamic pyruvic transaminase (GPT) activity.

Lactate dehydrogenase (LDH) activity and alkaline-phosphatase (ALP) activity, too, were measured with commercial UV kits (LDH-UV Test Wako; product of Wako Pure Chemical Industries, Ltd. and ALP-UV Test Wako; product of Wako Pure Chemical Industries, Ltd.), respectively. The final results of measurement are shown in Table 5.

TABLE 5

| | | Hercampuri-enriched |
|---|---|---|
| Factor tested | Control diet | diet |
| Body weight (g) | 313 ± 5 | 312 ± 7 |
| Food intake | 16.7 ± 0.8 | 15.8 ± 0.9 |
| (g/rat/day) | | |
| Blood pressure | 271 ± 3 | 258 ± 3** |
| (mmHg) | | |
| GOT (IU/L) | 75.2 ± 1.5 | 72.3 ± 2.0 |
| GPT (IU/L) | 21.8 ± 0.5 | 18.9 ± 1.9 |
| LDH (IU/L) | 215 ± 49 | 218 ± 44 |
| ALP (IU/L) | 582 ± 18 | 376 ± 36** |

**p (significant difference from the control) < 0.01;
Mean ± Standard error of the mean

As shown in Tables 3 through 5, there was no difference in the growth curve (increase in body weight) or the amount of food intake between the SHRSP fed with the diet containing Hercampuri powder and the SHRSP fed with the control diet. Both groups of rats grew favorably, proving the safety of either diet. With regard to the maximum blood pressure, the effectiveness of the diet containing Hercampuri powder in suppressing increase in the blood pressure was manifest in the 4th week and onward from the start of the feeding, and the final maximum blood pressure, which was measured after six weeks of feeding, was a significantly lower value.

As shown in Table 5, there was no difference in activities of the liver function-related serum enzymes, i.e. GOT, GPT, and LDH, between the Hercampuri-fed rats and the Control rats, which consisted of SHRSP fed with the control diet. Therefore, it can be concluded that intake of Hercampuri does not impair liver functions. However, compared with the Control rats, the Hercampuri-fed rats showed significantly lower values in ALP activity.

The cholesterol, phospholipid, and triglyceride contents in the serum were enzymatically measured with commercial kits (Cholesterol E-Test Wako, Phospholipid C-Test Wako, and Triglyceride E-Test Wako; products of Wako Pure Chemical Industries, Ltd.), respectively. Furthermore, thiobarbituric acid-reactive substances in the serum were measured with a commercial kit (peroxylipid-Test Wako; product of Wako Pure Chemical Industries, Ltd.). To be more specific, 1/12 Normal sulfuric acid and 10% phosphotangustic acid were added, and the mixture was thoroughly stirred.

The stirred serum then underwent centrifugation at 10000 rpm at 4° C. for 10 minutes. The resulting precipitate was mixed into distilled water so as to form a suspension. After adding a thiobarbituric acid (TBA) reagent to the suspension, reaction with the peroxylipid was allowed to progress in boiling water bath for 60 minutes. After cooling down the reaction substance, butanol was added to the cooled reaction substance in order to extract thiobarbituric acid-reactive substances (TBARS) therefrom. Thereafter, TBARS underwent centrifugation at 2500 rpm at 4° C. for 5 minutes, enabling the top layer to be separated and to measure its ability to withstand fluorescence with excitation wavelength of 515 nm and fluorescence wavelength of 515 nm.

In the same manner as above, the antioxidant property of low-density lipoprotein (LDL: bad cholesterol) was measured with a commercial kit (peroxylipid-Test Wako; product of Wako Pure Chemical Industries, Ltd.). To be more specific, the LDL fraction was dialyzed at 4° C. over a night by using 10 mM phosphate buffered saline (pH 7.4) and subsequently oxidized at 37° C. for four hours by means of 10 μM copper sulfate liquid. After adding EDTA/2Na, 1/12 N H₂SO₄ and, subsequently, 10% phosphotangustic acid were added, and the mixture was then thoroughly stirred. LDL-TBARS was then measured in the same manner as in the case of serum TBARS described above. The results of measurements are shown in Table 6.

TABLE 6

| | | Hercampuri-enriched |
|---|---|---|
| Serum concentration | Control diet | diet |
| Cholesterol (mg/dl) | 67.4 ± 3.0 | 72.4 ± 2.7 |
| Phospholipid | 156 ± 7 | 180 ± 8** |
| (mg/dl) | | |
| Triglyceride | 139 ± 10 | 137 ± 13 |
| (mg/dl) | | |
| TBARS (nmol MDA/mg | 6.77 ± 0.43 | 7.66 ± 0.82 |
| protein) | | |
| LDL-TBARS (nmol | 19.0 ± 0.9 | 17.3 ± 0.4 |
| MDA/mg protein) | | |

MDA: Malondialdehyde
**p (significant difference from the control) < 0.01;
Mean ± Standard error of the mean

As shown in Table 6, the results of measurement of serum lipids presented significantly high serum lipid contents in the Hercampuri-fed rats. Those high values were the result of the significantly high lipid content in the LDL fraction. Furthermore, significantly high cholesterol content was evident in the high-density lipoprotein (HDL: good cholesterol) fraction. Although no difference was evident in serum TBARS between the two groups, the Hercampuri-fed rats showed lower TBARS in the LDL fraction. Therefore, it can be surmised that these results substantiate Hercampuri's effectiveness in increasing HDL-cholesterol, i.e. good cholesterol, and the antioxidant property of LDL.

Immediately after the extraction of all the blood, the liver was quickly removed from all the rats in each group of SHRSP. After removing the surrounding connective tissues and wiping out the blood and the body fluids with a Kimwipes, the wet weight of each liver was measured. The liver weight ratio of each rat is shown in terms of the liver weight per unit of body weight (100 g) at the time of removal.

Extraction of lipid from each liver was performed in accordance with the method of Folch et al. (Folch et al., Journal of Biological Chemistry, vol. 226, p 497-509, 1957). To be more specific, after 1 ml of water was added to approximately 0.2 g of liver tissue under ice-cooling, the mixture was homogenized. Then, after adding 5 ml of a 2:1 chloroform-methanol liquid solution and continuing the homogenizing process, lipid was extracted. Thereafter, by means of centrifugation at 2500 rpm at 15° C. for 5 minutes, the processed solution was divided so that the lower layer, i.e. the chloroform-containing layer, was separated to be used as a liquid extract of the total liver lipid. A part of the liquid extract was separated, and the chloroform contained therein was evaporated under nitrogen gas current. The liquid with the chloroform removed was used as a sample for measuring liver lipid contents.

The cholesterol, phospholipid, and triglyceride contents in the liver were enzymatically measured with commercial kits (Cholesterol E-Test Wako, Phospholipid C-Test Wako, and Triglyceride E-Test Wako; products of Wako Pure Chemical Industries, Ltd.), respectively.

Furthermore, under ice-cooling, 9 ml of 50 mM tris-hydrochloric acid buffer (pH 7.4) containing 0.3 M of sucrose, 50 mM of common salt, 10 mM of EDTA-4Na, and 10 mM of dithiothreitol (DTT) was added to approximately 1 g of liver tissue, and the liquid mixture was homogenized by means of LABO-STIRRER LS-15 (product of Yamato Co., Ltd.). The homogenized mixture underwent centrifugation at 2500 rpm at 4° C. for 30 minutes to obtain supernatant fluid, which then underwent high-speed centrifugation at 16000 rpm at 4° C. for 20 minutes.

The resulting supernatant fluid underwent ultracentrifugation at 40000 rpm (100000 g) at 4° C. for 60 minutes to obtain precipitate, i.e. pellets, of the microsome fraction. 4 ml of microsome extracting buffer was added to the precipitate. After the mixture was formed into a suspension at 4° C., 40000 rpm (100000 g) ultracentrifugation was performed again at 4° C. for 60 minutes to obtain dark brown precipitate, which was used as liver microsome fraction. Thereafter, 500 μl of phosphoric-acid buffer was mixed in, and the mixture was stirred under ice-cooling with a multi-stirrer until the precipitate completely dissolved. The resulting suspension was stored at −80° C. to be used for measurement of activities of enzymes related to liver lipid metabolism.

With the cholesterol in the liver microsome fraction serving as a substrate, 7 α-hydroxy cholesterol generated from oxidization, i.e. hydroxylation reaction using NADP system resulting from cholesterol 7α-hydroxylase (7α-hydroxylase), was converted into 7α-hydroxy-4-cholesten-3-on (HCO) by means of cholesterol oxidase. After the conversion reaction was completed, the reaction product (HCO) was extracted with n-hexane and analyzed by HPLC in accordance with the method of Ogishima et al. (Ogishima and Okuda; Analytical Biochemistry, vol. 158, p 228, 1986).

The results of measurement of 7α-hydroxylase activity are shown in Table 7 in terms of the amount of HCO generated per 1 mg of protein content in one minute in the microsome fraction (pmol/min/mg of microsome protein).

TABLE 7

| | | Hercampuri-enriched |
|---|---|---|
| Concentration in liver | Control diet | diet |
| Liver weight | 3.90 ± 0.05 | 3.86 ± 0.12 |
| (g/100 g B.W.) | | |
| Cholesterol (mg/wet g) | 10.8 ± 0.8 | 8.81 ± 0.40* |
| Phospholipid | 31.3 ± 0.7 | 33.6 ± 1.4 |
| (mg/wet g) | | |
| Triglyceride | 57.4 ± 7.4 | 46.2 ± 4.8 |
| (mg/wet g) | | |
| 7α-hydroxylase | 9.9 ± 0.9 | 15.0 ± 1.5* |
| (nmol/min/mg protein) | | |

*p (significant difference from the control) < 0.01;
Mean ± Standard error of the mean

As shown in Table 7, there was no significant difference in liver weight per unit of body weight between the two test groups. Regarding the lipid content in per unit of liver weight, however, the Hercampuri-fed rats manifested significantly low cholesterol content and slightly low triglyceride content. Furthermore, the Hercampuri-fed rats manifested significantly high values in the activity of cholesterol 7α-hydroxylase, which is a rate-limiting enzyme in catabolism of liver cholesterol to bile acid. It can be surmised from these results that the diet containing Hercampuri extract powder accelerated cholesterol 7α-hydroxylase activity and thereby increased the catabolism of liver cholesterol to bile acid and its discharge into bile, resulting in the low liver cholesterol content.

As described above, raising SHRSP with diet containing dried Hercampuri or alcohol-extract of whole dried Hercampuri plants not only enabled SHRSP to grow favorably but also showed such effects as superior suppression of increase in blood pressure, suppression of bad cholesterol, and increasing good cholesterol. It has thus been ascertained that Hercampuri (its extract powder) is effective in maintaining or improving health.

(Test 3)

Next, an explanation is given of a test to determine the inhibitory activity of Hercampuri on human salivary α-amylase.

Among numerous medical effects of Hercampuri, it can be surmised that antiobesity effect and antidiabetic effect are attributed to Mangiferin, which is one of the xanthone derivatives contained in Hercampuri. Therefore, the inhibitory activity of Hercampuri on the starch decomposition activity of human salivary α-amylase was studied by using Hercampuri extract and pure Mangiferin.

Samples used for this experiment were prepared from undiluted Hercampuri extract (provided by TOWA Corporation), soluble starch (product of Wako Pure Chemical Industries, Ltd.), and 10000 units/ml of α-amylase (from human saliva: product of Lee Scientific, Inc.)

α-amylase is an enzyme existing in human saliva and pancreatic juice and has the function of hydrolysing starch into dextrin, maltotriose, maltose, and isomaltose. Should the activity of α-amylase be inhibited, hydrolysis of starch is prevented. In other words, digestion and absorption of starch is suppressed, resulting in antidiabetic effect and antiobesity effect.

How sample solutions were prepared is now described. First, ethanol contained in the undiluted Hercampuri extract was evaporated by means of an evaporator, and the remaining liquid was frozen at −80° C. and subsequently freeze dried. 10 ml of 80% ethanol was added to 10 mg of the resulting powder (measured to the nearest 0.1 mg) to dissolve the powder completely. Thereafter, by diluting the solution with 80% ethanol, sample solutions having different concentrations, i.e. 10, 20, 50, 100, 200, and 500 μg/ml, were produced. The sample solutions were then stored at 5° C.

In the same manner as above, 10 ml of 50% ethanol was added to 1 mg of Mangiferin (measured to the nearest 0.1 mg) to dissolve the powder completely. Thereafter, by diluting the solution with 50% ethanol, sample solutions having different concentrations, i.e. 1, 2, 5, 10, 20, and 50 μg/ml, were produced. The sample solutions were then stored at 5° C.

Soluble starch solution has to be prepared each time a test is performed. The preparation process consists of measuring 200 to 500 mg of soluble starch (measured to the nearest 0.1 mg), adding 5 ml of purified water, heating the solution in boiling water bath for 10 minutes, and thoroughly stirring the solution. After being cooled to room temperature, the solution can be used.

The process of preparing α-amylase solution consists of taking 10 μl from commercially available α-amylase solution (10000 units/ml), mixing in 0.25 M phosphoric-acid buffer (pH 7.0) in order to increase the volume to 10 ml, dividing the solution into smaller portions of 1 ml each, and freeze-storing them at −20° C. To use, the solution should be diluted to a desired concentration with 0.25 M phosphoric-acid buffer (pH 7.0). Other necessary reagents should be prepared in accordance with normal methods.

Next, how the inhibitory activities on the α-amylase from human saliva were measured is explained hereunder.

Twenty five μl of each sample solution was mixed into its own corresponding 50 μl soluble starch solution, with each mixture under going preincubation at 37° C. for 5 minutes. Twenty five μl of α-amylase solution was added 20 seconds after the end of preincubation, and reaction was allowed to progress at 37° C. for 30 minutes.

The reaction of the samples was halted by mixing 1 ml of 0.1 M hydrochloric acid into each sample 20 seconds after completion of the 30-minute incubation of each sample so as to obtain 200 μl each of reaction solution. After putting 2 ml of 0.01 M iodine solution, each reaction solution was thoroughly stirred and left untouched for one hour. Thereafter, the absorbance of each reaction solution was measured at 660 nm. The absorbance of each reaction solution of the Control group, which had been prepared in the same manner as above, was also measured at 660 nm.

The enzyme inhibitory ratio of each Hercampuri sample solution was calculated in comparison with each respective Control sample. The calculation was performed based on the equation Enzyme Inhibitory ratio (%) [(C−B)/(A−B)]×100, wherein A is starch solution+buffer; B is starch solution+enzyme solution+buffer; and C is starch solution+enzyme solution+sample solution.

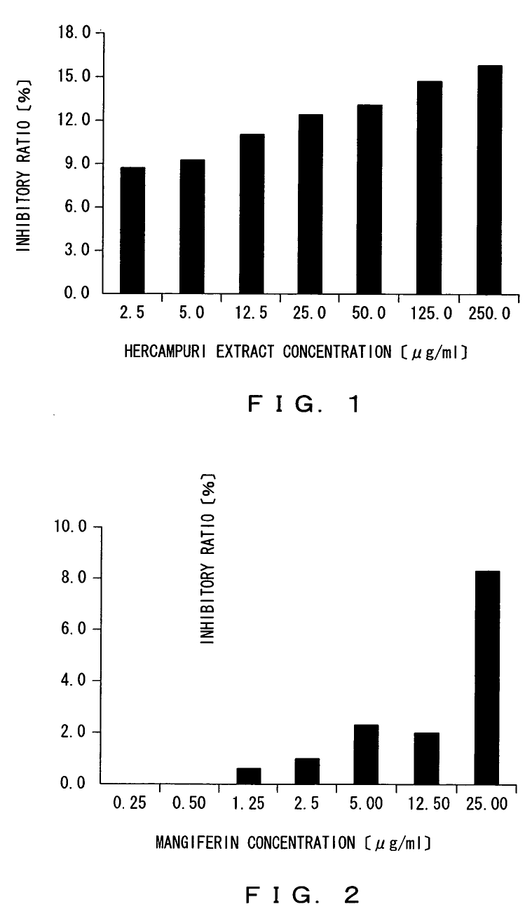

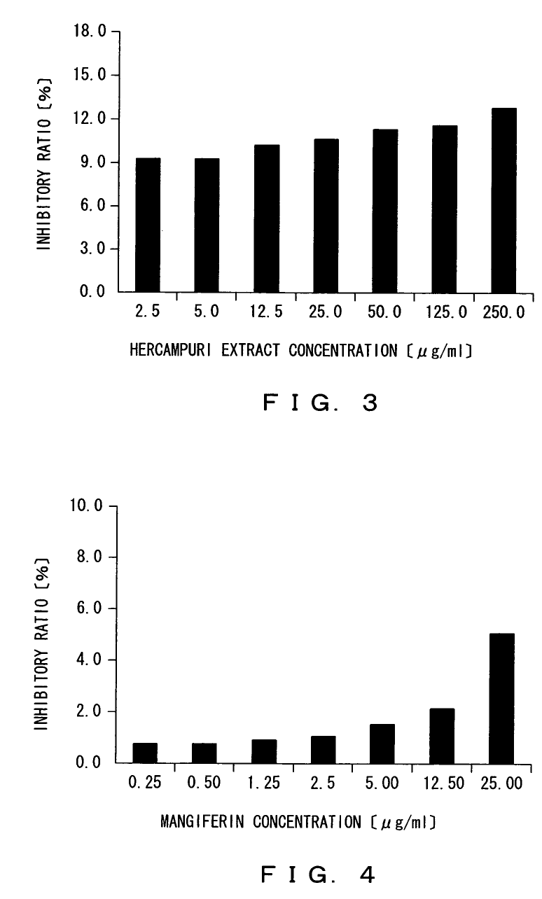

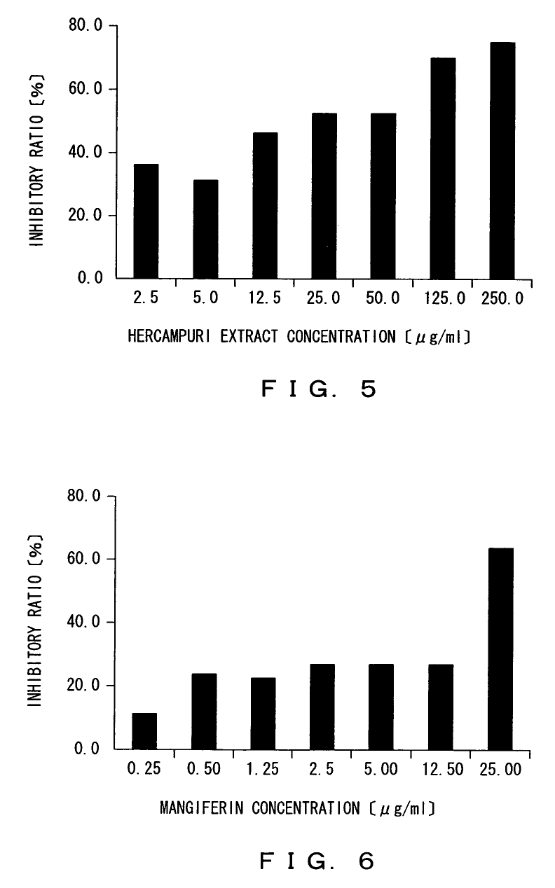

As described above, the inhibitory ratios of various solutions on α-amylase activity were measured, and the results of measurement shown in FIGS. 1 through 6 suggest that of the three kinds of reaction systems, both Mangiferin and Hercampuri extract have the function of inhibiting enzyme activities. In particular, the high concentration samples manifested distinct inhibitory activity. However, when the enzyme concentration was 1 unit/ml, the degree of hydrolysis was too high (from 80 to 90%) with the samples having a substrate concentration of 2 mg/ml and 5 mg/ml, resulting in a relatively low overall inhibitory ratio. In FIGS. 1 through 6, the values on the horizontal axis represent the final concentrations of the solutions of each reaction system.

When the enzyme concentration and the substrate concentration were 0.5 unit/ml and 5 ml/ml respectively, the degree of hydrolysis was low (from 20 to 50%), resulting in a high overall inhibitory ratio. Therefore, it can be surmised that further adjustment of the concentration ratio between the substrate and the enzyme will produce even better results.

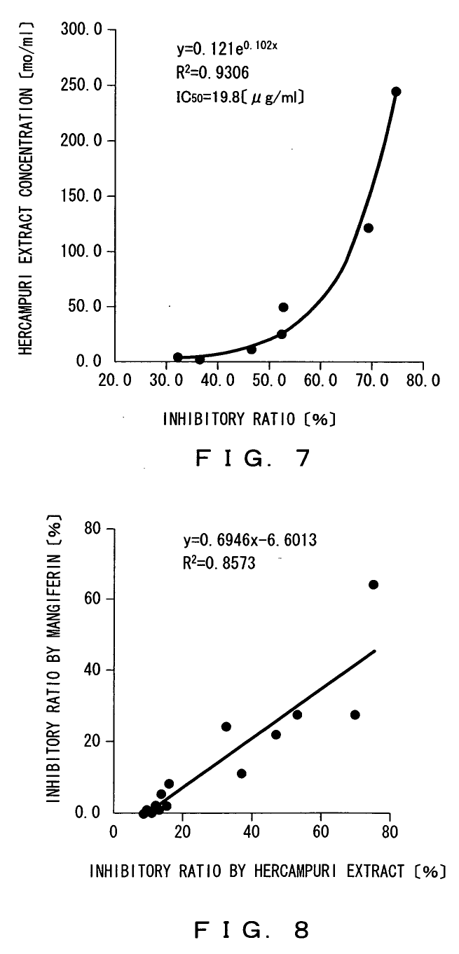

As shown in FIG. 7, which represents the correlation between the concentrations of and the inhibitory activities by Hercampuri extract, the samples in the reaction system with the enzyme concentration of 0.5 unit/ml and the substrate of 5 ml/ml showed 50% enzyme inhibitory activity when Hercampuri content (IC₅₀) was 19.8 μg/ml.

As is evident from the correlation between the inhibitory activities of Hercampuri extract and Mangiferin, which is manifested in the results obtained through tests on the three reaction systems and shown in FIG. 8, the inhibitory activities of the two different reaction systems produced a linear correlation. As described previously, a better regression equation may be found through reaction systems with an adjusted concentration ratio between the substrate and the enzyme. In other words, these results suggest the possibility of establishing a method of estimating the Mangiferin content in Hercampuri extract based on a regression equation.

Therefore, it has been proved in vitro that Hercampuri extract inhibits α-amylase activity. In the reaction system with the enzyme concentration and the substrate of 0.5 unit/ml and 5 ml/ml respectively, the intensity of inhibitory activity (IC₅₀) was approximately 20 μg/ml. This result substantiated antidiabetic effect and antiobesity effect of Hercampuri extract.

As the aforementioned inhibitory activity exhibited a high correlation with the inhibitory activity of Mangiferin, it can be surmised that the inhibitory activity on the α-amylase by Hercampuri extract principally resulted from the activity of Mangiferin. Furthermore, the high correlation between the inhibitory activities of Hercampuri extract and the amount of Mangiferin has verified that the Mangiferin content in Hercampuri extract can be estimated by measuring the inhibitory activity on the α-amylase by Hercampuri extract. It has thus been confirmed that it is possible to establish a method of evaluating the quality of Hercampuri extract.

(Test 4)

Next, an explanation is given of a test to determine the effectiveness of Hercampuri in treating diabetes.

By using the α-glucosidase fraction obtained from rat intestinal tracts, the inhibitory activity by Hercampuri extract powder on the α-glucosidase was studied.

First of all, to separate crude α-glucosidase fraction, a 300 to 400 g male Wister rat was euthanized with ether anesthesia. The jejunum portion of the rat (to be more specific, the portion from the duodenal suspensory ligament to between 20 and 25 cm downward) was then extracted and sliced into pieces approximately 3 cm in length under ice-cooling. Then, after each piece was incised and thoroughly washed with ice-cooled physiological saline solution, a mucous membrane portion was carefully scraped off with a slide glass. The obtained mucous membrane portion was suspended in ice-cooled 2 mM tris-hydrochloric acid buffer solution (pH 7.1) containing 50 mM Mannitol and homogenized at a low speed under ice-cooling. Thereafter, 0.5 M calcium chloride is diluted to achieve a 50-fold increase in the volume so as to make the final concentration 10 mM was added, and the solution was left untouched for 15 minutes under ice-cooling.

Thereafter, the solution underwent low-speed centrifugation at 5500 rpm (3000 g) at 4° C. for 15 minutes to obtain supernatant fluid, which then underwent high-speed centrifugation at 16400 rpm (27000 g) at 4° C. for 30 minutes. The precipitate of the brush-border-membrane fraction resulting from this high-speed centrifugation was suspended in 0.1 M maleic acid buffer solution (pH 6.0). At that time, 500 μl of 0.1 M maleic acid buffer solution (pH 6.0) was used for the precipitate of the brush-border-membrane fraction obtained from each rat. With distilled water, 5 μl of the aforementioned suspension solution was diluted to 100 μl, of which 40 μl was used to measure the protein content. The content of protein was measured to be in the range of 4 mg/ml to 6 mg/ml. The aforementioned suspension solution was diluted with 0.1 M maleic acid buffer solution (pH 6.0) in order to achieve a 10-fold increase in the volume and subsequently stored in a frozen state at −40° C. until such time that it is used.

Next, how the inhibitory activities on the α-glucosidase were measured is explained hereunder. A number of solutions were prepared, each solution consisting of 20 μl of 74 mM sucrose or maltose added to 10 μl of Hercampuri extract sample, which was a solution diluted to an appropriate concentration with 0.1 M maleic acid buffer solution (pH 6.0), and each mixture solution underwent preincubation at 37° C. for 3 minutes. Thus, solutions respectively with different Hercampuri concentrations were prepared. Then, 10 μl of the solution prepared by diluting the crude α-glucosidase fraction solution that had been obtained in the manner described above with 0.1 M maleic acid buffer solution (pH 6.0) in order to achieve a 10-fold increase in the volume was added to each preincubated sample solution, and the mixture solution subsequently underwent incubation at 37° C. for 30 minutes.

After 160 μl of distilled water was added to each incubated reaction solution so as to increase the volume to 200 μl, each diluted reaction solution was stirred and submerged in boiling water bath for 2 minutes. After cooling these reaction solutions rapidly, a part (40 μl) of each reaction solution was used to determine the quantity of glucose generated therein. The measurement was conducted by using Glucose CII-Test Wako (product of Wako Pure Chemical Industries, Ltd.). Twenty five μl of 0.1 M maleic acid buffer solution (pH 6.0) was used as the control sample with 0% inhibitory ratio.

The measurement of inhibitory activities of Mangiferin on the α-glucosidase is explained hereunder. Mangiferin used for this measurement was produced by dissolving 2.112 mg of Xanthone C-glucoside (Sigma M3547) in 4 ml of 50% MeOH. This Mangiferin was diluted with 0.1 M maleic acid buffer solution (pH 6.0) to produce solution samples having respective concentrations of 1 mM, 0.5 mM, 0.25 mM, 0.125 mM, and 0.063 mM. Twenty μl of 74 mM sucrose or maltose was added to 10 μl of each solution sample, and the mixture solution underwent preincubation at 37° C. for 3 minutes.

Ten μl of the solution prepared by diluting the crude α-glucosidase fraction solution that had been obtained in the manner described above with 0.1 M maleic acid buffer solution (pH 6.0) in order to achieve a 10-fold increase in the volume was added to each preincubated sample solution, and the mixture solution subsequently underwent incubation at 37° C. for 30 minutes. After 160 μl of distilled water was added to each incubated reaction solution so as to increase the volume to 200 μl, each diluted reaction solution was stirred and submerged in boiling water bath for 2 minutes. After cooling these reaction solutions rapidly, a part (40 μl) of each reaction solution was used to determine the quantity of glucose generated therein. The measurement was conducted by using Glucose CII-Test Wako (product of Wako Pure Chemical Industries, Ltd.).

It is evident from the results of the measurements that inclusion of Hercampuri diluted from extract at a ratio of 1:200 to 1:300 results in 50% inhibitory activity (IC₅₀) on the α-glucosidase.

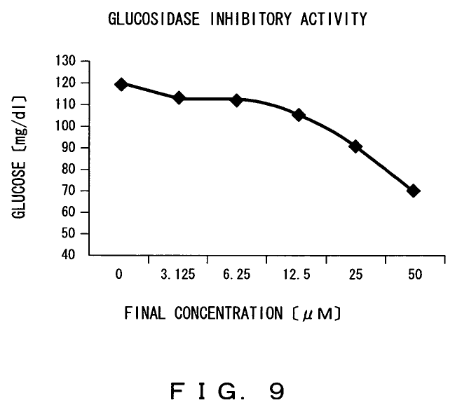

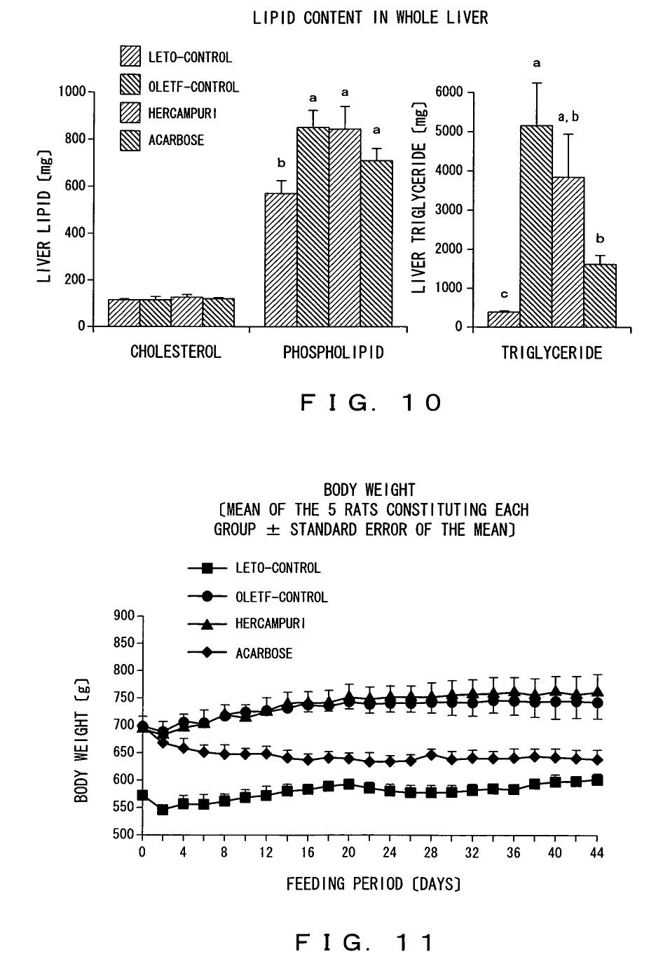

As shown in FIG. 9, IC₅₀ of Mangiferin was measured to be in the range of 0.1 mg/ml to 0.15 mg/ml. Furthermore, IC₅₀ of carbose, which is a product of Bayer AG and used as an ntidiabetic drug at present, was measured in the same manner s above, with the results of measurements being in the range f 0.12 μg/ml to 0.15 μg/ml.

It is widely known that Bellidifolin is another xanthone derivative contained in Hercampuri extract. Among the xanthone derivatives contained in Hercampuri, Bellidifolin is found in the largest amounts. It is also known that Bellidifolin has a function of reducing blood sugar levels in streptozotocin (STZ)-induced diabetic rats. As is evident from the above description, Hercampuri extract has a high content of xanthone derivatives that are known to be effective in improving blood sugar levels, in other words, curative treatment of diabetes, and possesses significant potential for use as an antidiabetic agent.

### PRODUCT EXAMPLE 1

0.01 g of orange flavoring and 10 g of potato starch were mixed with 1 g of Hercampuri extract powder obtained through Test 1, and tablets, i.e. a food product, were produced from the mixture in accordance with a normal method.

### PRODUCT EXAMPLE 2

Ten grams (10 g) of refined white sugar and 0.05 g of orange flavoring were mixed with 0.5 g of Hercampuri extract powder obtained through Test 1. By adding water to this mixture in order to make the total volume 120 ml, and packaging it in a plastic bottle, a health drink, i.e. a beverage product, was produced.

Next, a diabetes controlling composition according to a second embodiment of the present invention and containing Hercampuri is explained hereunder.

The diabetes controlling composition according to the invention is effective in controlling diabetes, in particular, Type 2 diabetes. Another feature of the diabetes controlling composition lies in that it contains, at least, Hercampuri (Gentianella alborosea (Gilg) Fabris). Hercampuri is a perennial dicotyledon belonging to the gentianales order of the Gentianaceae family that grows in the cold climate of the high altitude plains called puna, in the Peruvian Andes, 3500 to 4000 m above sea level.

The diabetes controlling composition contains Hercampuri in the form of Hercampuri extract powder, which is produced by adding aqueous alcohol, i.e. 70% alcohol, to dried whole Hercampuri plant (roots, stems, and leaves) at a mixing ratio of 5 parts aqueous alcohol to 1 part dried Hercampuri to produce by immersion a Hercampuri extract solution, to which dextrin is added, and then spray-drying the mixture to obtain Hercampuri extract powder. This Hercampuri extract powder has a high content of xanthone derivatives.

The diabetes controlling composition according to the invention is effective in controlling Type 2 diabetes. In other words, it has functions of improving serum and liver lipid conditions, lowering serum glucose levels, improving free fatty acid levels, reducing insulin resistance, and decreasing cholesterol levels. The diabetes controlling composition is well-suited for use in functional foods, cosmetics, skin preparations for external use, and pharmaceuticals.

The aforementioned diabetes controlling composition may be provided in any form selected from among powder, granule, tablet, sugar-coated tablet, capsule, liquid, and syrup. Whichever form is chosen, the product may be prepared with an auxiliary or a flavor additive. Examples of the excipient or the diluent that can be used include gelatin, various saccharides, starch, fatty acids, their salts, fats and oils, talc, physiological saline, and other masking agents. Although the product formulated as above may be ingested as is, it may be conveniently ingested in various prepared foods, confectioneries, or candies.

### ACTUAL EXAMPLE

An explanation is given hereunder regarding what influence administration of Hercampuri extract powder may exert on metabolism of sugar and lipids at the onset of diabetes symptoms.

An experiment was conducted to study the influence exerted y administration of Hercampuri extract powder on Type 2 diabetes in comparison with Acarbose, which is an antidiabetic drug. The experiment was conducted using Otsuka Long-Evans Tokushima Fatty (OLETF) rats, which are an animal model of Type 2 diabetes, and Long-Evans Tokushima Otsuka (LETO) rats.

OLETF rats mentioned above are a new strain of rat with non-insulin dependent diabetes mellitus, i.e. Type 2 diabetes. OLETF rats are widely known as an animal model of Type 2 diabetes and allow for stepwise control of diabetic symptoms.

OLETF rats are a strain that have spontaneously high blood sugar levels with diabetic symptoms. Furthermore, OLETF rats have characteristic features that enable the observation throughout the process of their developing diabetic symptoms, from when they are young and have not yet developed insulin resistance to the period during which insulin resistance becomes apparent and to the onset of diabetic symptoms.

LETO rats are a control animal for comparison with OLETF rats. Although LETO rats are a strain from which OLETF rats were developed, LETO rats are not prone to diabetes.

First, the materials and the method used for the experiment are explained.

(Materials)

Hercampuri extract powder used as a subject sample of the experiment is an extract powder that is a product of TOWA Corporation and is produced by adding aqueous alcohol, i.e. 70% alcohol, to dried whole Hercampuri plant (roots, stems, and leaves) at a mixing ratio of 5 parts aqueous alcohol to 1 part dried Hercampuri to produce by immersion a Hercampuri extract solution, to which dextrin is added, and then spray-drying the mixture to obtain Hercampuri extract powder. The content of extract in the extract powder was 60% by mass.

Mangiferin content, which is an indicator of the total amount of xanthone compounds contained in the extract powder was in the range of 8.0 to 8.7% (measured by the HPLC method). For the HPLC method (High Performance Liquid Chromatography), HPLC reagents (product of Wako Pure Chemical Industries, Ltd.) were used. Furthermore, cholesterol oxidase (product of Asahi Kasei Corporation) was also used. Other necessary reagents were selected from among special grade reagents manufactured by Wako Pure Chemical Industries, Ltd.

(Preparation of Diet)

The rats that were the subjects of the experiment were divided into three groups: a Control group to be fed a control diet, a Hercampuri group to be fed a Hercampuri-enriched diet, and an Acarbose group to be fed an Acarbose-enriched diet. The control diet had an AIN-93 composition described in “AIN-93 purified diets from rodents” by Reeves and two other members in Journal of Nutrition (USA, vol. 123, P.1923-1931, 1993). This control diet was purified animal feed containing casein as the source of protein. The Hercampuri-enriched diet fed to the rats in the Hercampuri group was produced by adding Hercampuri extract powder at a mixing ratio of 2% to the control diet. The Acarbose-enriched diet fed to the rats in the Acarbose group was produced by adding Acarbose (product of Bayer AG) at a mixing ratio of 0.2% to the control diet. For each experimental diet, the amount of the added subject ingredient was compensated for by adjusting the amount of corn starch. The respective compositions of the two types of experimental diet and the control diet are shown in Table 8.

TABLE 8
Composition of experimental diet

| | The | The | The |
|---|---|---|---|
| | Control | Hercampuri | Acarbose |
| Ingredients | group | group | group |
| Hercampuri Ext. powder | — | 2.00 | — |
| Acarbose | — | — | 0.20 |
| Milk casein | 20.00 | 20.00 | 20.00 |
| L-Cystine | 0.30 | 0.30 | 0.30 |
| Soybean oil | 10.00 | 10.00 | 10.00 |
| Mineral mixture (AIN-93G) | 3.50 | 3.50 | 3.50 |
| NB1 | | | |
| Vitamin mixture (AIN-93) | 1.00 | 1.00 | 1.00 |
| Cellulose powder | 5.00 | 5.00 | 5.00 |
| Corn starch | 36.75 | 34.75 | 36.55 |
| α-Corn starch | 13.20 | 13.20 | 13.20 |
| Sucrose | 10.00 | 10.00 | 10.00 |
| Choline bitartrate | 0.25 | 0.25 | 0.25 |

[Percent by mass]

(Subject Animals and Method for Raising them)

Male OLETF rats and LETO rats delivered from Tokushima Research Institute of Otsuka Pharmaceutical Co., Ltd. were used as the subject animals. Each one of these OLETF rats and LETO rats was born during the period of June 10th to June 13th in 2002. Until the experiment was initiated, these OLETF rats and LETO rats had been kept in an animal room at a temperature of 22.0±2.0° C. and humidity of 55.0±5.0% with 12-hour changeover of the ambient light (the light period being from 7:00 am to 7:00 pm and the dark period from 7:00 pm to 7:00 am). The rats had free access to feed (CE-2: product of Clea Japan, Inc.) and drinking water (tap water).

Thereafter, 33-week old OLETF rats in which the presence of glucose in the excreted urine had been ascertained with Pretest 3aII (product of Wako Pure Chemical Industries, Ltd.) and 33-week old LETO rats were selected to be used for the experiment. In other words, five 33-week old rats were selected from each of the OLETF and LETO groups to be used as controls: the OLETF rats were used as controls for tests for effectiveness in curative treatment of diabetes and the LETO rats were used as the normal rat controls.

Furthermore, each of the Hercampuri-fed group and the Acarbose-fed group consisted of 33-week old five OLETF rats. These OLETF rats and LETO rats were kept for six weeks under the same conditions as before the experiment except that each group was allowed free access to feed on each respective type of food and drinking water (tap water). During the experiment, the body weights and the amounts of food intake were measured every other day. The experiment was performed in accordance with Animal Experiment Guideline of Kinki University School of Medicine.

(Collecting Whole Blood and Separation of Serum)

Six week after the initiation of the experiment, without prior abstinence from feed, each rat was anesthetized by administering approximately 50 mg/kg of pentobarbital sodium (product of Dainippon Pharmaceutical Co., Ltd.) into the abdominal cavity to remove all of its blood via the abdominal aorta with an injection syringe. This operation was performed during the period from 10:00 am to 12:00 am. The blood obtained by the operation was set aside for 1 hour at room temperature and subsequently at 4° C. for a period in the range of 1 to 2 hours. Thereafter, blood serum was separated by means of centrifugation at 2500 rpm at 4° C. for 10 minutes.

(Separation of Lipoprotein Fractions from Blood Serum)

Fractions of various lipoproteins were separated from the blood serum obtained at the time of whole blood extraction. The lipoprotein fractions separated in this stage were very-low-density lipoprotein (VLDL; d<1.006) fraction, low-density lipoprotein (LDL; d:1.006-1.063) fraction, high-density lipoprotein (HDL; d:1.063-1.210) fraction, and lipo-free (Free; d>1.210) fraction.

At that time, the separation of each lipoprotein fraction was performed by means of step-wise density gradient ultracentrifugation (TL-100; product of Beckman Coulter Inc., USA) with a fixed-angle rotor (TLA-100.2; product of Beckman Coulter Inc., USA) in accordance with “Practical method for plasma lipoprotein analysis” by F. T. Hatch (Academic Press, USA, vol. 1, p. 1-68, 1968).

However, as described in “Isolation and partial characterization of high-density lipoprotein-1 (HDL₁) from rat plasma by gradient centrifugation” by L. T. Lusk and three other members (USA, Biochemical Journal, vol. 183, P83-89, 1979), it is known that, with OLETF rats and LETO rats, a sub-fraction of HDL, i.e. HDL₁, is present across the range of the LDL fraction and the HDL fraction. Therefore, it is not possible with step-wise density gradient ultracentrifugation employed for this experiment to exclusively separate native LDL, and the LDL fraction separated in this stage contains HDL₁ that existed together with LDL.

(Measurement of Cholesterol, Phospholipid, and Triglyceride Content in Each Lipoprotein Fraction and Serum, and Measurement of Glucose and Free Fatty Acid Content in Serum)

The cholesterol, phospholipid, and triglyceride contents in the serum and each lipoprotein fraction were enzymatically measured with commercial kits (Cholesterol E-Test Wako, Phospholipid C-Test Wako, and Triglyceride E-Test Wako; products of Wako Pure Chemical Industries, Ltd.), respectively.

Furthermore, the glucose and free fatty acid content in he serum were enzymatically measured with commercial kits (Glucose CII-Test Wako and NEFA C-Test Wako; products of Wako Pure Chemical Industries, Ltd.), respectively.

(Measurement of Serum Insulin Content)

The serum insulin content was determined in accordance with the Sandwich ELISA (enzyme-linked immunosorbent assay) method using a Rat insulin ELISA kit (product of Shibayagi Co., Ltd.). Therefore, the insulin content is shown in terms of Immuno-reactive insulin (IRI; pg/ml).

(Measurement of Various Apolipoprotein Contents in Serum)

The respective contents of various apolipoproteins, i.e. apoA-I, apoB, and apoE, as well as the respective apoE contents in the LDL and HDL fractions, were determined by the rocket immunoelectrophoresis method described in “Age-related changes in serum concentrations of apolipoprotein A-I, E, and A-IV in SHRSP” by Hiroshi Ogawa and 4 other members (G.B., Journal of Hypertension, vol. 4, p 429-453, 1986). The aforementioned immunoelectrophoresis method is in accordance with the Laurell method and adjustment of a part of the method presented by C. B. Laurell in “Quantitative estimation of proteins by electrophoresis in agarose-gel containing antibodies” (USA, Analytical Biochemistry, vol. 15, p 42-52, 1966). The antibodies used for this measurement were specific antibodies obtained from rabbits by using antigens that were various apolipoproteins separated and refined from rat serum. Furthermore, apoB/apoA-I was calculated from the results of measurement of apoA-I and apoB.

(Removal of Liver, Measurement of Liver Weight Ratio, and Extraction of Liver Lipid)

Immediately after the extraction of all the blood, the liver was quickly removed from all the rats in each group, and the wet weight of each liver was measured. The liver weight ratio of each rat is shown in terms of the liver weight per unit of body weight (100 g) at the time of removal. Extraction of lipid from each liver was performed in accordance with the method described in “A simple method for the isolation and purification of total lipids from animal tissues” (Folch and two other members, Journal of Biological Chemistry, vol. 226, p 497-509, 1957). A part of the lower layer of the liquid liver extract (the chloroform-containing layer) was separated, and the chloroform contained therein was evaporated under nitrogen gas current. The liquid with the chloroform removed was used as a sample for measuring liver lipid contents.

(Measurement of Cholesterol, Phospholipid, and Triglyceride Content in Liver)

The cholesterol, phospholipid, and triglyceride contents in the liver were enzymatically measured with commercial kits (Cholesterol E-Test Wako, Phospholipid C-Test Wako, and Triglyceride E-Test Wako; products of Wako Pure Chemical Industries, Ltd.), respectively.

(Measurement of the Activity of 7α-Hydroxylase, Rate-Limiting Enzyme in the Synthesis of Bile Acid)

Purified liver microsome fraction was produced according to a normal method, and the cholesterol in the liver microsome fraction was used as the substrate. The activity of cholesterol 7α-hydroxylase was determined by HPLC in accordance with the method described in “An improved method for assay of cholesterol 7α-hydroxylase activity” by Tadashi Ogishima and one other member (USA, Analytical Biochemistry, vol. 158, p 228-232, 1986). The results of measurement of 7α-hydroxylase activity are shown in terms of the amount of HCO (7α-hydroxy-4-cholesten-3-on) generated per 1 mg of protein content in one minute in the microsome fraction (pmol/min/mg of microsome protein).

(Statistical Processing)

All the experimental values are shown in terms of the mean±standard error of the mean (M±SE). As for test of significant difference, after performing analysis of variance by using StatView (Ver. 5, SAS Institute Inc., USA), Tukey's multiple comparison test was performed. A risk ratio of 5% or less was considered significant.

Next, the results of the experiment performed with the materials and the method described above are explained.

(Influence on Body Weights and Food Intake)

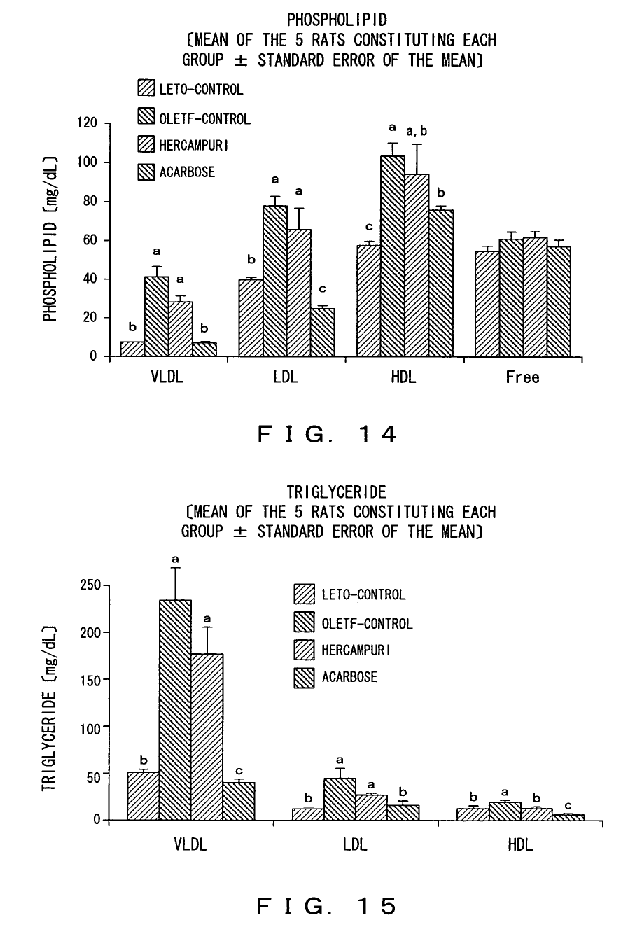

Changes during the experiment in body weights and amounts of food intake of the rats of each one of the four groups, i.e. the two control-diet groups (the LETO rat group and the OLETF rat group), the Hercampuri-fed rat group, and the Acarbose-fed rat group, are shown in FIGS. 11 and 12. As shown in FIGS. 11 and 12, the OLETF rats were already significantly heavier in body weight than the LETO rats when the experiment started. Such a significant difference continued until the experiment ended. The Hercampuri-fed rats and the OLETF rats showed similar changes in body weight. However, from the 6th day of the start of the administration of the Acarbose-enriched diet, the Acarbose-fed rats began to show significantly lower weights than did the OLETF rats and continued to show such significantly lower body weights until the administration was terminated.

The amount of food intake by the OLETF rats already significantly exceeded that by the LETO rats when the experiment started. Such a significant difference continued until the experiment ended. The Hercampuri-fed rats and the OLETF rats showed similar changes. The Acarbose-fed rats began to show significantly lower food intake compared with the OLETF rats until the 12th day of the start of the administration of the Acarbose-enriched diet. Thereafter, however, their food intake increased. As a result, the amount of food intake by the Acarbose-fed rats remained greater than that by the OLETF rats from the 20th day until the last day of the administration.

(Influence on Serum Contents of Cholesterol, Phospholipid, and Triglyceride)

Final body weights at the end of the experiment, average amounts of food intake, and the respective serum contents of cholesterol, phospholipid, and triglyceride obtained at the end of the experiment are shown in Table 9.

TABLE 9

| Diet | Control | Control | Hercampuri | Acarbose |
|---|---|---|---|---|
| Rat strain | LETO | OLETF | OLETF | OLETF |
| Body weight | 599 ± 10^{c} | 744 ± 34^{a} | 761 ± 33^{a} | 639 ± 14^{b} |
| (g) | | | | |
| Food intake | 9.37 ± 0.50^{b} | 12.8 ± 0.6^{a} | 13.3 ± 0.9^{a} | 12.9 ± 0.9^{a} |
| (g/rat/day) | | | | |
| Cholesterol | 145 ± 5^{b} | 235 ± 11^{a} | 209 ± 30^{a,b} | 150 ± 11^{b} |
| (mg/dL) | | | | |
| Phospholipid | 171 ± 6^{b} | 314 ± 16^{a} | 277 ± 36^{a} | 178 ± 10^{b} |
| (mg/dL) | | | | |
| Triglyceride | 68.8 ± 5.2^{b} | 320 ± 51^{a} | 219 ± 36^{a} | 55.6 ± 1.7^{c} |
| (mg/dL) | | | | |

As shown in Table 9, the LETO rats alone showed significantly low final body weights at the end of the experiment and low average food intake compared with the other three groups. While the Acarbose-fed rats showed significantly lower final body weights than did the OLETF rats, no significant difference was ascertained between the Hercampuri-fed rats and the OLETF rats.

In the respective serum contents of cholesterol, phospholipid, and triglyceride obtained at the end of the experiment, the Acarbose-fed rats showed values that were significantly lower than those of the OLETF rats and at nearly the same level as those of the LETO rats. On the other hand, while the values shown by the Hercampuri-fed rats were lower than those of the OLETF rats, the values were not significantly lower.

(Influence on Serum Contents of Glucose, Free Fatty Acids, and Insulin)

Respective serum contents of glucose, free fatty acids, and insulin are shown in Table 10.

TABLE 10

| Diet | Control | Control | Hercampuri | Acarbose |
|---|---|---|---|---|
| Rat strain | LETO | OLETF | OLETF | OLETF |
| Glucose | 172 ± 8^{c} | 390 ± 36^{a} | 312 ± 13^{b} | 193 ± 12^{c} |
| (mg/dL) | | | | |
| FFA | 828 ± 53^{c} | 2268 ± 231^{a} | 1605 ± 235^{b} | 751 ± 38^{c} |
| (μEq/L) | | | | |
| IRI | 642 ± 147^{c} | 4335 ± 1407^{a} | 3635 ± 1317^{a} | 665 ± 231^{b} |
| (pg/mL) | | | | |

As shown in Table 10, in the serum contents of glucose, the Acarbose-fed rats showed values that were significantly lower than those of the OLETF rats and at nearly the same level as those of the LETO rats. The Hercampuri-fed rats showed values lower (p<0.08) than those of the OLETF rats. In the respective serum contents of free fatty acids and insulin, the Acarbose-fed rats showed values that were significantly lower than those of the OLETF rats and at nearly the same level as those of the LETO rats. On the other hand, while the values shown by the Hercampuri-fed rats were lower than those of the OLETF rats, the values were not significantly lower.

(Influence on Various Apolipoprotein Contents in Serum)

The respective serum contents of various apolipoproteins obtained at the end of the experiment, i.e. apoA-I, apoB, and apoE, as well as apoB/apoA-I, are shown in Table 11.

TABLE 11

| Diet | Control | Control | Hercampuri | Acarbose |
|---|---|---|---|---|
| Rat strain | LETO | OLETF | OLETF | OLETF |
| apoA-I | 67.5 ± 3.0^{b} | 119 ± 8^{a} | 114 ± 16^{a} | 126 ± 4^{a} |
| (mg/dL) | | | | |
| apoB | 5.39 ± 0.17^{a} | 4.44 ± 0.27^{b} | 4.04 ± 0.16^{b} | 3.95 ± 0.14^{b} |
| (mg/dL) | | | | |
| apoE | 72.6 ± 2.0^{a} | 56.4 ± 3.0^{b} | 55.3 ± 2.4^{b} | 55.5 ± 2.7^{b} |
| (mg/dL) | | | | |
| ApoB/ | 8.16 ± 0.41^{a} | 3.79 ± 0.22^{b} | 3.80 ± 0.49^{b,c} | 3.15 ± 0.06^{c} |
| apoA-I | | | | |
| (×100) | | | | |

As shown in Table 11, the LETO rats showed significantly low serum contents of apoA-I compared with the other three groups, while no significant difference was ascertained among the other three groups. In contrast to the case of apoA-I contents, the LETO rats showed significantly high values in the respective serum contents of apoB and apoE compared with the rats of the other three groups, while no significant difference was ascertained among the rats of the other three groups. The LETO rats showed significantly higher values in apoB/apoA-I compared with the rats of the other three groups, while the Acarbose-fed rats showed significantly lower values than those of the OLETF rats. Furthermore, virtually no difference was seen between the Hercampuri-fed rats and the OLETF rats.

(Influence on Contents of Cholesterol, Phospholipid, and Triglyceride in Each Serum Lipoprotein Fraction)

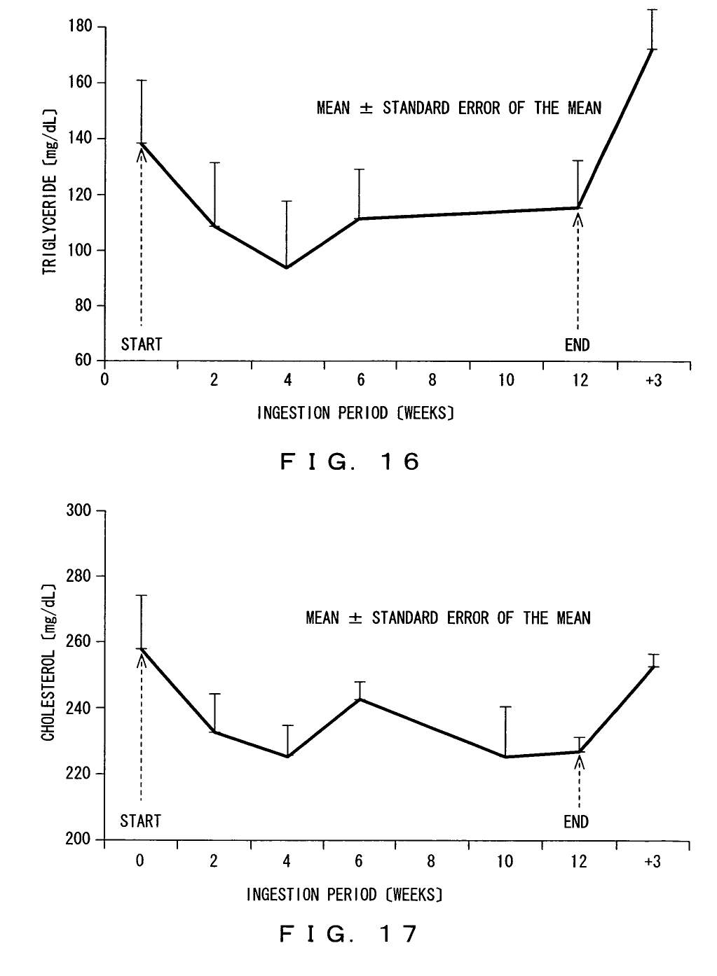

The distribution of the respective contents of cholesterol, phospholipid, and triglyceride in each serum lipoprotein fraction are shown in FIGS. 13 through 15.

The cholesterol contents in serum lipoprotein fractions are shown in FIG. 13. As shown in FIG. 13, the Acarbose-fed rats showed values that were, compared with those of the OLETF rats, significantly lower in both the VLDL fraction and the LDL fraction, and compared with those of the LETO rats, at nearly the same level in the VLDL fraction and significantly lower in the LDL fraction. On the other hand, while the values shown by the Hercampuri-fed rats were lower than those of the OLETF rats, the values were not significantly lower. In the HDL fraction, the LETO rats showed significantly low values compared with the rats of the other three groups, while no significant difference was ascertained among the rats of the other three groups.

As shown in FIG. 14, the distribution of the phospholipid contents in serum lipoprotein fractions was relatively similar to the distribution of the cholesterol contents. To be more specific, the Acarbose-fed rats showed values that were, compared with those of the OLETF rats, significantly lower in both the VLDL fraction and the LDL fraction, and compared with those of the LETO rats, at nearly the same level or significantly lower. On the other hand, while the values shown by the Hercampuri-fed rats were lower than those of the OLETF rats, the values were not significantly lower. In the HDL fraction, the LETO rats showed significantly low values compared with the rats of the other three groups, while the values shown by the Acarbose-fed rats were lower than those of the OLETF rats. Furthermore, while the values shown by the Hercampuri-fed rats were lower than those of the OLETF rats, the values were not significantly lower. In the lipo-free fraction, virtually no difference was seen among the four groups.

The triglyceride contents in serum lipoprotein fractions are shown in FIG. 15. As shown in FIG. 15, the Acarbose-fed rats showed values that were, compared with those of the OLETF rats, significantly lower in both the VLDL fraction and the LDL fraction. In comparison with those of the LETO rats, the Acarbose-fed rats showed values that were significantly lower in the VLDL fraction and at nearly the same level in the LDL fraction. Furthermore, while the values shown by the Hercampuri-fed rats were lower than those of the OLETF rats, the values were not significantly lower. In the HDL fraction, the values shown by the Acarbose-fed rats were significantly lower than those of the OLETF rats as well as the LETO rats, while the values shown by the Hercampuri-fed rats were lower than those of the OLETF rats.

(Influence on Liver Weight Ratio as Well as on Contents of Cholesterol, Phospholipid, and Triglyceride in Liver)

The liver weight ratio as well as contents of cholesterol, phospholipid, and triglyceride in liver are shown in Table 12.

TABLE 12

| Diet | Control | Control | Hercampuri | Acarbose |
|---|---|---|---|---|
| Rat strain | LETO | OLETF | OLETF | OLETF |
| Liver weight | 2.92 ± 0.02^{c} | 40.3 ± 0.16^{a} | 3.59 ± 0.23^{a,b} | 3.40 ± |
| (g/100 g B.W.) | | | | 0.09^{b} |
| Cholesterol | 6.37 ± 0.60^{a} | 3.71 ± 0.39^{c} | 4.67 ± 0.33^{b} | 5.39 ± |
| (mg/wet g) | | | | 0.37^{a,b} |
| Phospholipid | 32.5 ± 3.3 | 28.4 ± 2.2 | 30.5 ± 1.8 | 32.9 ± |
| (mg/wet g) | | | | 2.4 |
| Triglyceride | 22.8 ± 2.0^{c} | 167 ± 29^{a} | 132 ± 26^{a} | 75.4 ± |
| (mg/wet g) | | | | 8.2^{b} |
| 7α-hydroxylase | 13.4 ± 0.5^{c} | 21.5 ± 2.6^{b} | 25.6 ± 1.4^{a,b} | 34.5 ± |
| (nmol/min/mg | | | | 5.7^{a} |
| protein) | | | | |

The LETO rats showed significantly low liver weight ratio compared with the other three groups, while the values shown by the Acarbose-fed rats were lower than those of the OLETF rats. Furthermore, while the values shown by the Hercampuri-fed rats were lower than those of the OLETF rats, the values were not significantly lower. In contrast to the case of liver weight ratio, the LETO rats showed significantly high values in the cholesterol content in liver compared with the rats of the other three groups. The Acarbose-fed rats showed significantly higher values than those of the OLETF rats. The Hercampuri-fed rats showed values higher (p<0.09) than those of the OLETF rats.

Regarding the phospholipid content in liver, no significant difference was ascertained among the rats of the four groups. The LETO rats showed significantly low triglyceride content in liver compared with the other three groups, while the values shown by the Acarbose-fed rats were lower than those of the OLETF rats. Furthermore, while the values shown by the Hercampuri-fed rats were lower than those of the OLETF rats, the values were not significantly lower.

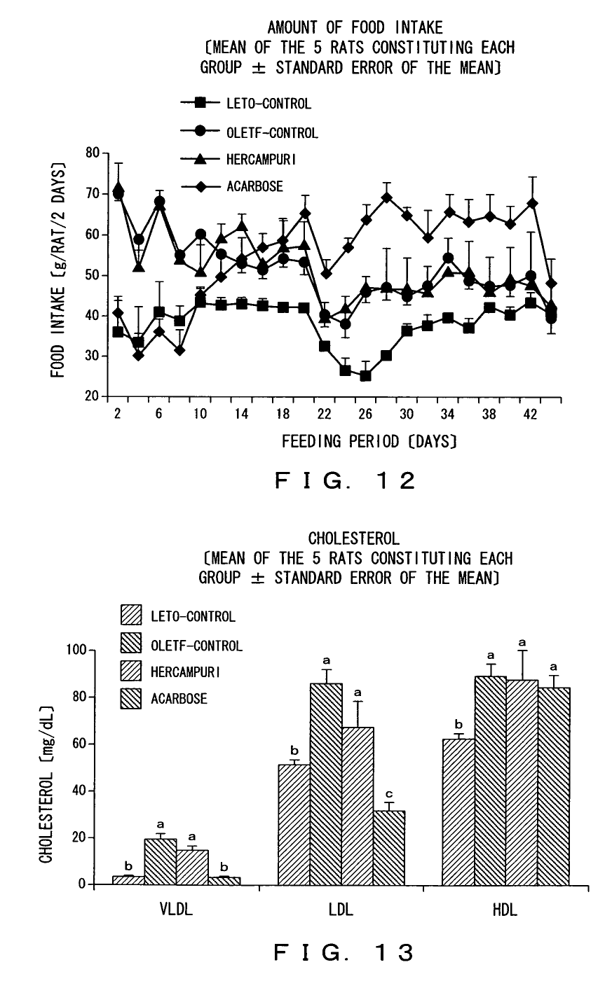

The amount of lipid contained in whole liver of each group of rats is shown in FIG. 10. As shown in FIG. 10, regarding the cholesterol content in whole liver, no significant difference was ascertained among the rats of the four groups. The LETO rats showed significantly low phospholipid content in whole liver compared with the other three groups, while no significant difference was ascertained among the other three groups.

Furthermore, the LETO rats showed significantly low triglyceride content in whole liver compared with the other three groups. The values shown by the Acarbose-fed rats were lower than those of the OLETF rats. While the values shown by the Hercampuri-fed rats were lower than those of the OLETF rats, the values were not significantly lower.

(Influence on Activity of 7α-Hydroxylase in Microsome fraction in the Liver)

The activity of 7α-hydroxylase in microsome fraction in the liver is shown in Table 12.

As shown in Table 12, the LETO rats showed significantly low values in the activity of 7α-hydroxylase compared with the other three groups, while the Acarbose-fed rats showed values higher (p<0.08) than those of the OLETF rats. While the values shown by the Hercampuri-fed rats were higher than those of the OLETF rats, the values were not significantly higher.

(Observation)

The Japan Diabetes Society provides a general definition of diabetes as a disorder where the blood sugar level on an empty stomach is higher than 7.0 mmol/L (126 mg/dL) or the blood sugar level two hours after 75 g OGTT (Oral Glucose Tolerance Test) is higher than 11.1 mmol/L (200 mg/dL). Recently, hemoglobin Alc levels (HbAlc) are also used for diagnosis of diabetes. HbAlc, which is also called glycohemoglobin, results from glucose bonding itself to hemoglobin. Therefore, glycohemoglobin is not easily affected by food and clearly reflects blood sugar levels of the recent four months. Normally, when HbAlc is more than 6.5%, a diagnosis of diabetes is made.

There are two types of diabetes: Type 1 diabetes resulting from insufficiency in the absolute amount of insulin and Type 2 diabetes resulting from insufficiency in the relative amount of insulin primarily caused by insulin resistance. The majority of Japanese diagnosed with diabetes suffer from Type 2 diabetes. In recent years, drastic changes in Japanese lifestyles, including the Westernization of dietary habits, have produced environmental factors leading to a dramatic increase in people with Type 2 diabetes. In many cases, Type 2 diabetes is associated with obesity, insulin resistance, hyperlipidemia, or hypertension. Although it is evident that these diseases are closely connected with one another, the details of the correlation remain largely unknown.

As described above, according to the present embodiment, an experiment was conducted to study the influence exerted by administration of Hercampuri extract powder, which is rich in xanthone derivatives, on Type 2 diabetes in comparison with Acarbose, which is an antidiabetic drug. The experiment was conducted using OLETF rats, which are widely known as an animal model of Type 2 diabetes, and the LETO rats, which are a control animal for comparison

As a result of the experiment, virtually no difference was seen in the growth curve or the amount of food intake between the Hercampuri-fed rats and the LETO rats. However, the body weight of the Acarbose-fed rats showed significant decrease, until finally reaching the same level as that of the LETO rats, while the amount of food intake increased. It is possible that Acarbose has a strong effect on the reduction of the food intake efficiency resulting from the α-glucosidase inhibitory activity.

Regarding influence on serum lipids, the Acarbose-fed rats showed very strong improvements in all the respective serum contents of cholesterol, phospholipid, and triglyceride, showing nearly the same values as those of the LETO rats. Furthermore, detailed study of distribution of each lipid in serum lipoprotein substantiated Acarbose's significant capability of reducing VLDL and LDL in the OLETF rats.

On the other hand, Acarbose produced no significant difference in HDL; the Acarbose-fed rats maintained significantly high HDL compared with the LETO rats. The Hercampuri-fed rats showed improvement, showing values lower than those of the OLETF rats in all the serum lipid contents. In distribution of each lipid in serum lipoprotein, too, the Hercampuri-fed rats manifested similar effects to those of the Acarbose-fed rats, with reduced VLDL and LDL, while showing no significant difference in HDL and maintaining significantly higher HDL than the LETO rats. The aforementioned HDL condition is substantiated by the fact that the LETO rats alone showed significantly lower values than those shown by the rats of the other three groups in the apoA-I content, which is the principal protein component of HDL, while no significant difference was ascertained among the rats of the other three groups.

The effectiveness of Acarbose and Hercampuri in improving serum lipid conditions is also substantiated by the fact that both the Acarbose-fed rats and the Hercampuri-fed rats showed values somewhat lower than those shown by the OLETF rats in the apoB content, which is the principal protein component of VLDL and LDL. However, no definite result was obtained for apoE, which is the principal protein component of VLDL and HDL₁. Therefore, it has been substantiated that administration of Hercampuri extract powder is effective in improving serum lipid conditions of the OLETF rats, which is an effect similar to that produced by Acarbose. The effect produced by Hercampuri extract powder, however, was weaker than that of Acarbose.

Influence on serum glucose, free fatty acids and insulin is now discussed. Acarbose reduced serum glucose of the OLETF rats to nearly the same level as that of the LETO rats, and Hercampuri, too, considerably reduced levels of serum glucose. For free fatty acids and insulin, too, Acarbose reduced free fatty acids and insulin in the serum of OLETF rats to nearly the same level as those of the LETO rats, and Hercampuri was also capable of reducing the levels to a certain level. Therefore, as is true with the effect on serum lipids, administration of Hercampuri was effective in improving such serum conditions as glucose, free fatty acids, and insulin levels of the OLETF rats. The effect produced by Hercampuri extract powder, however, was weaker than that of Acarbose.

Next, regarding on influence on the liver, Acarbose significantly suppressed a significant increase in liver weight per unit of body weight and a significant increase in triglyceride content (mg/wet g) in the OLETF rats. Although Hercampuri exhibited similar effects to those of Acarbose, those effects were not significant, and were weaker than those of Acarbose. However, both Acarbose and Hercampuri significantly suppressed decrease in the cholesterol content (mg/wet g) in the OLETF rats. This suppression went hand in hand with increase in the activity of 7α-hydroxylase, which is a rate-limiting enzyme in catabolism of liver cholesterol to bile acid. Regarding the cholesterol content in whole liver, however, no significant difference was ascertained among the rats of the four groups, and homeostasis was sufficiently maintained.

Regarding the phospholipid content (mg/wet g), no significant difference was ascertained among the rats of the four groups. As for lipid content in whole liver, however, the LETO rats alone showed significantly low values, while no difference was ascertained among the rats of the other three groups. Therefore, it was judged that both Acarbose and Hercampuri exerted only a small influence on lipid content in the liver of OLETF rats. From these results, it has been ascertained that Hercampuri has similar effect to that of Acarbose in liver lipid metabolism as well. This effect, however, was weaker than that of Acarbose.

Xanthone derivatives are known to be among the principal components of Hercampuri extract powder. It is also known that Bellidifolin and Mangiferin, both of which are xanthone derivatives, have a function of reducing blood sugar levels and increasing insulin sensitivity in an animal model of diabetes. Therefore, it is highly probable that the effects of Hercampuri extract powder on the OLETF rats are attributable to the functions of such xanthone derivatives as Bellidifolin and Mangiferin.

As an overall assessment, the effects of Hercampuri extract powder manifested in the present experiment were weak, compared with those of Acarbose. The Mangiferin content, which is an indicator of the total amount of xanthone derivatives contained in Hercampuri extract powder used for the experiment, was in the range of 8.0 to 8.7%. Therefore, Mangiferin content in the Hercampuri-enriched diet for the experiment was in the range of 0.16 to 0.174%, i.e. approximately 0.8 times the Acarbose content in the Acarbose-enriched diet for the experiment. Taking this into consideration, in addition to the fact that the in vitro α-glucosidase inhibitory activity (ID₅₀) of Mangiferin is approximately 1/100 that of Acarbose (determined by Department of Hygiene, Kinki University School of Medicine), it can be judged that Hercampuri extract powder produced adequate results.

Furthermore, as Hercampuri extract powder is classified as a food, the function of Hercampuri extract powder as a functional food product should be mild; it should not be as strong as Acarbose, which is a therapeutic. Therefore, it can be surmised from this viewpoint that the antidiabetic function of Hercampuri extract powder, which is a new function found by the present experiment, has a potential to be highly useful in a wide range of application.

To summarize, it can be judged that Hercampuri extract powder has controlling functions, such as functions of preventing or curative treatment of what is widely known as the metabolic syndrome, i.e. an interlinked complex of diseases resulting from accumulation of visceral fat. Therefore, Hercampuri extract powder can be used as a composition for controlling disorders induced by excessive calorie intake.

Next, a fat reducing composition according to a third embodiment of the present invention and containing Hercampuri is explained hereunder.

The fat reducing composition according to the invention is effective in lowering total cholesterol in serum and decreasing triglycerides in serum, lowering blood pressure, and reducing subcutaneous fat as well as visceral fat. Another feature of the fat reducing composition lies in that it contains, at least, Hercampuri.

The fat reducing composition is also effective in preventing or alleviating risk factors for metabolic syndrome, which will be explained later. The abovementioned metabolic syndrome is what is widely known as metabolism disorder syndrome, in other words a condition caused by a combination of risk factors. Metabolic syndrome is also referred to as “syndrome X”, “the deadly quartet”, or “multiple risk factor clustering”.

To be more specific, metabolic syndrome means a condition with a plurality of lifestyle-related diseases that could develop into atherosclerosis, in other words a condition with a complex of lifestyle-related diseases. A person with metabolic syndrome is subject to a substantially higher risk of progress of atherosclerosis, and is therefore prone to such diseases as cerebral infarction, angina pectoris, or myocardial infarction. The abovementioned risk factors for metabolic syndrome are obesity, hyperlipidemia, hypertension, and diabetes, of which obesity is a prerequisite. Metabolic syndrome is a condition where a person suffers from obesity and at least two of the other risk factors.

The definition of the abovementioned risk factors for metabolic syndrome are different between Japan and the revised Adult Treatment Panel III (ATP III of 2001) put out by the National Cholesterol Education Program (NCEP) of the United States.

In Japan, obesity is defined as a condition where the circumference of the abdomen at the height of the navel (waist circumference) is greater than 85 cm for men and 90 cm for women; hyperlipidemia is defined as a condition where the triglyceride (TG) measurement is higher than 150 mg/dl, or HDL cholesterol (so-called good cholesterol) measurement is lower than 40 mg/dl or both; hypertension is defined as a condition where the systolic pressure is higher than 130 mmHg, or the diastolic pressure is higher than 85 mmHg or both; and diabetes is defined as a condition where the blood sugar level on an empty stomach is higher than 110 mg/dl, which is a level for borderline diabetes.

NCEP-ATP III Guidelines (2001) formulated in the United States defines obesity as a condition where the circumference of the abdomen at the height of the navel (waist circumference) is greater than 102 cm for men and 88 cm for women; hyperlipidemia is defined as a condition where the triglyceride (TG) measurement is higher than 1.7 m mol/l, or HDL cholesterol (so-called good cholesterol) measurement is lower than 1.0 m mol/l for men (1.3 m mol/l for women) or both; hypertension is defined as a condition where the systolic pressure is higher than 130 mmHg and/or the diastolic pressure is higher than 85 mmHg, or a condition where the person is under medication to lower blood pressure; and diabetes is defined as a condition where the blood sugar level on an empty stomach is higher than 6.1 m mol/l.

The aforementioned fat reducing composition effective in preventing or alleviating risk factors for metabolic syndrome is well-suited for use in functional foods, cosmetics, skin preparations for external use, and pharmaceuticals. The fat reducing composition is characterized in that it contains Hercampuri extract powder, which is powder extracted from Hercampuri, and that the composition is effective in preventing or alleviating metabolic syndrome. The composition having characteristics such as those mentioned above is used in a food or beverage product, pharmaceuticals or the like, with a label indicating its use for prevention or alleviation of metabolic syndrome.

### ACTUAL EXAMPLE

An explanation is given hereunder regarding an actual example showing what influence the ingestion of Hercampuri extract powder may exert on risk factors for metabolic syndrome.

(Subjects of the Experiment)

The subjects of the experiment were healthy individuals who lived healthy lives conducting normal daily activities. Although some of them were found in a physical checkup to be Close to the borderline for having one or more of the risk factors for metabolic syndrome, none of them was taking medication. A total of 12 individuals consisting of ten men and two women participated in the experiment as the test diet group. The experiment was conducted in strict accordance with the ethical principles and testing protocol set forth in the Declaration of Helsinki. Prior to commencement of the experiment, the subjects were provided with sufficient and thorough explanation regarding various details, including the nature of the research, the method of the experiment, and the possible side effects that could be anticipated, and submitted of their free will in writing their respective consent.

(Food Sample for Testing)

Tablets produced from Hercampuri extract powder (PREMIEX®), each tablet containing approximately 150 mg of Hercampuri extract powder, were used as food sample for testing.

(Experiment Schedule and Manner of Ingestion)

The duration of the experiment was a total of 18 weeks consisting of three periods: a 3-week non-ingestion period as a preliminary survey period, a 12-week ingestion period, and another 3-week non-ingestion period as a postliminary survey period. During each non-ingestion period, blood was taken from each subject three hours after a meal. Once every two weeks during the ingestion period, blood was taken from each subject three hours after a meal. Furthermore, alcohol and smoking were strictly forbidden prior to taking blood samples.

As to the manner of the ingestion of the aforementioned food sample for testing, the ingestion schedule called for each female subject to take a total of 6 to 8 tablets per day and each male subject to take a total of 8 to 10 tablets per day, with each subject being instructed to take the tablets twice a day, within 30 minutes of eating breakfast and within 30 minutes of eating dinner. Because the food sample for testing had a bitter taste, a certain latitude was allowed in the dosage so that each subject could adjust the number of tablets to take within the aforementioned range. Other than taking food sample for testing, the subjects were instructed to continue the same daily routine they had prior to taking part in the experiment, including their dietary habits, smoking habits, exercise regime, etc. Furthermore, a daily record was kept by the subjects to keep track of food and beverage consumption, smoking, alcohol, and physical conditions.

(Measurement Method)

Each subject measured his or her own blood pressure and pulse once a week, at a predetermined time during the preliminary survey period, the ingestion period, and the postliminary survey period. The measurement of blood pressure and pulse was performed in accordance with WHO/ISH Hypertension Guidelines; for each subject, after the subject had been at rest in a sitting position for 5 minutes, the blood pressure and the pulse were measured 3 times at intervals of a few minutes, and the averages of the measured values were respectively recorded as the blood pressure and pulse.

As for physical measurements and clinical laboratory test, each subject measured his or her own height, body weight, BMI, basal metabolic rate, body fat ratio, muscle mass, and V/S (visceral fat-to-subcutaneous fat ratio) ratio once a week, at a predetermined time. Regarding hematological test and biochemical test of the extracted blood, Clinical Laboratory of Kinki University Hospital was requested to perform such examinations.

The examination consisted of 18 categories for serum analysis, i.e. TP, Alb, GOT, GPT, γ-GTP, LDH, Glu, ALP, Na, K, Ca, Cl, BUN, CRN, UA, T-Cho, TG, and HDL-Cho, and 5 factors for blood cell counting, i.e. RBC, WBC, Hb, Ht, and Plt.

Next, the results of the experiment on the actual example described above are explained.

During the period when the experiment was underway, no subject dropped out of the experiment after commencing the ingestion of the food sample for testing or for any reasons resulting from the ingestion of the food sample for testing. Furthermore, the ingestion exerted no unfavorable influence on activities of the liver function-related serum enzymes.

There were three subjects who exhibited relatively high serum triglyceride measurement (more than 130 mg/dl) at the commencement of the experiment, of which two exhibited a lowering in their respective serum triglycerides. The serum triglyceride level is easily influenced by diet and therefore prone to fluctuation. Nevertheless, as shown in FIG. 16, it can be concluded that the ingestion of tablets produced from Hercampuri extract powder had a favorable effect of decreasing triglyceride levels in subjects with relatively high triglyceride levels.

As for the total cholesterol in serum, there were a total of eight subjects with elevated total cholesterol from the beginning, in other words, those who exhibited abnormally high total cholesterol level (more than 220 mg/dl) at the commencement of the experiment and those with total cholesterol at the high end of normal. All of these eight subjects exhibited a decrease in their respective total cholesterol levels after six weeks of the ingestion of the food sample for testing. After 12 weeks of the ingestion of the food sample for testing, six of the aforementioned eight subjects exhibited a gentle decrease in their respective total cholesterol levels. Therefore, as shown in FIG. 17, it can be concluded that there is a strong possibility that the ingestion of tablets produced from Hercampuri extract powder had a favorable effect of lowering total serum cholesterol in subjects with relatively high total serum cholesterol

As for the blood pressure, there were a total of five subjects with borderline high blood pressure, of which three exhibited a lowering in either one of or both systolic and diastolic blood pressure. Blood pressure is subject to large circadian variation and also prone to varying widely depending on the method of measurement. Nevertheless, it can be concluded that there is a possibility that the ingestion of tablets produced from Hercampuri extract powder had a favorable effect on subjects with borderline high blood pressure in lowering their blood pressure.

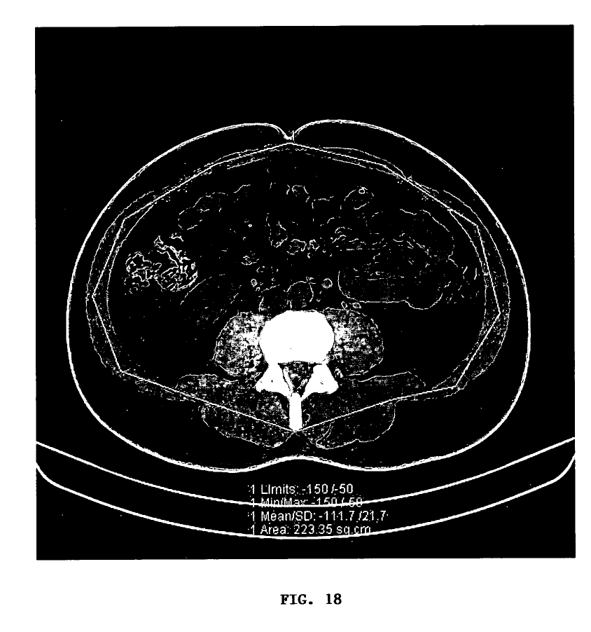

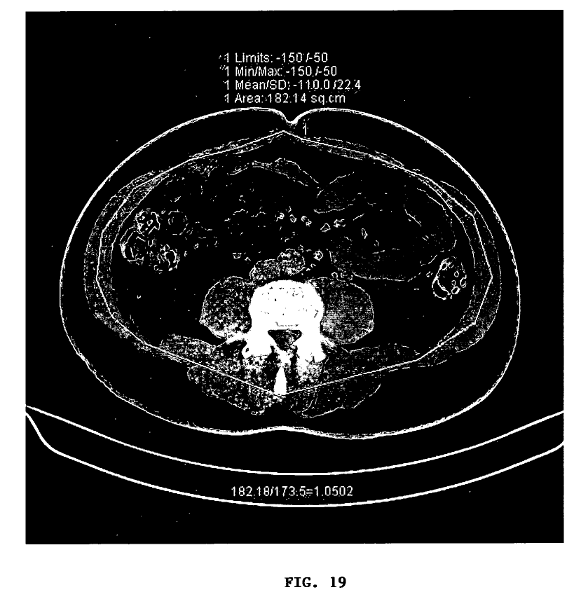

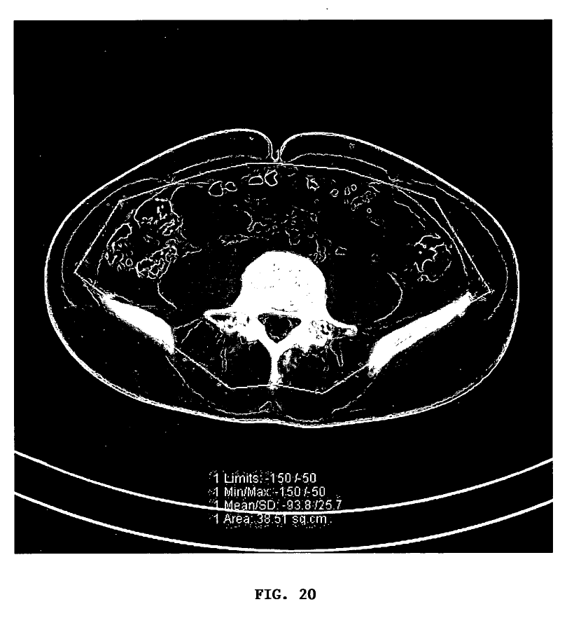

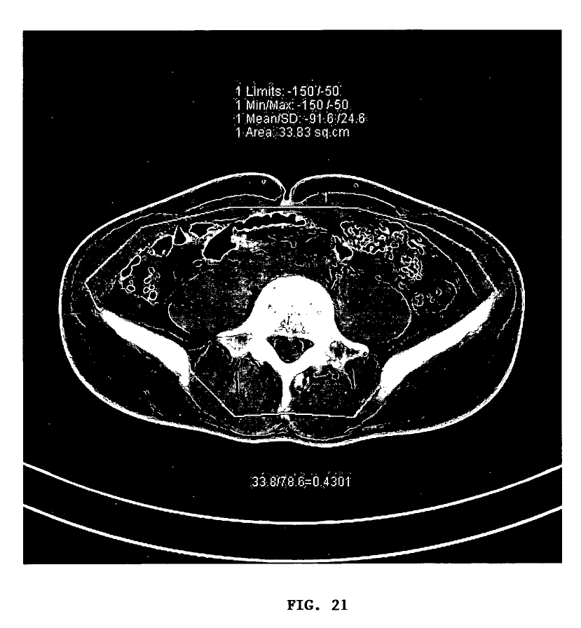

Scanning by means of a computerized tomography diagnostic equipment (Multislice CT) was performed on three subjects to measure the area of their visceral and subcutaneous fat of each subject at the beginning and end of the ingestion period. As shown in FIGS. 18 and 19, one of the three subjects, viz. Subject A, exhibited within a period of approximately three months a decrease in the area of the visceral fat from 223 cm² to 182 cm², in other words a decrease in visceral fat by 18.4%. As shown in FIGS. 20 and 21, another subject, viz. Subject B, exhibited within a period of approximately three months a decrease in the area of the visceral fat from 38.5 cm to 33.8 cm² and the area of the subcutaneous fat from 94.3 cm² to 78.5 cm², in other words decreases in visceral fat by 12.2% and subcutaneous fat by 16.8%. It was thus observed that the ingestion of tablets produced from Hercampuri extract powder is effective in lowering total cholesterol in serum and decreasing triglycerides in serum, lowering blood pressure, and reducing visceral fat. Therefore, it can be concluded that the ingestion of tablets produced from Hercampuri extract powder is effective in preventing or alleviating metabolic syndrome.

To summarize, it can be judged that tablets produced from Hercampuri extract powder have controlling functions, such as functions of preventing or alleviating what is widely known as metabolic syndrome, i.e. an interlinked complex of diseases resulting from accumulation of visceral fat. Therefore, tablets produced from Hercampuri extract powder can be used as a composition for controlling disorders induced by excessive calorie intake.

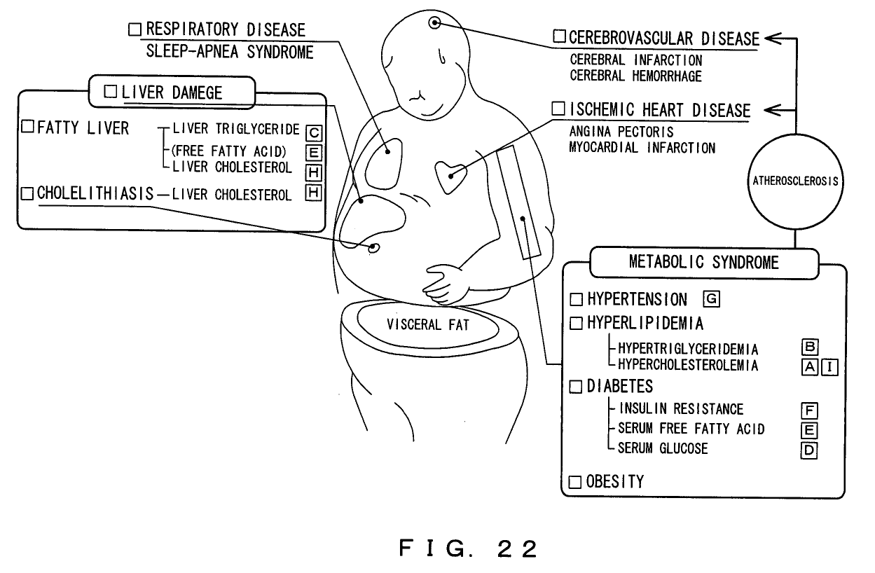

As shown in FIG. 22, the aforementioned metabolic syndrome means a condition caused by a combination of lifestyle-related disorders, such as hypertension, hyperlipidemia, diabetes, and obesity, caused by accumulation of visceral fat. The hyperlipidemia mentioned above includes hypertriglyceridemia, hypoalphalipoproteinemia, and hypercholesterolemia. Diabetes is closely associated with insulin resistance, serum free fatty acids, and increase in serum glucose levels.

By preventing or treating atherosclerosis through prevention or treatment of the aforementioned metabolic syndrome, it is possible to prevent or treat various disorders, including respiratory diseases, such as sleep-apnea syndrome, fatty liver or cholelithiasis resulting from increase in triglyceride or cholesterol in the liver, cerebrovascular diseases, such as cerebral infarction or cerebral hemorrhage, and ischemic heart diseases, such as angina pectoris or myocardial infarction.

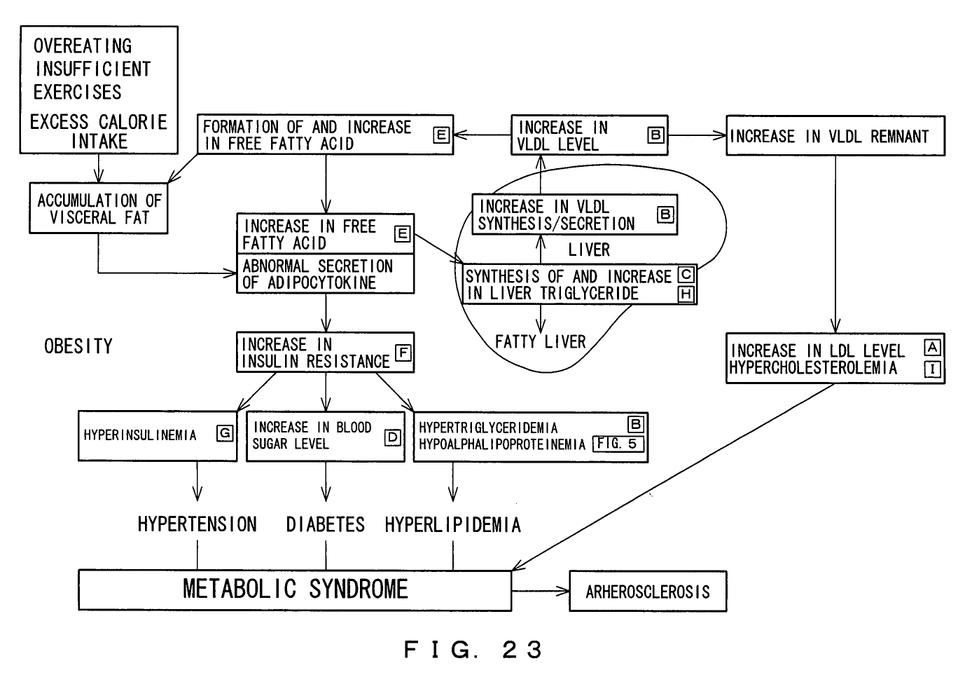

To be more specific, as shown in FIG. 23, obesity is induced by accumulation of visceral fat caused by excessive calorie intake resulting from various reasons, such as overeating or insufficient exercises. Accumulation of visceral fat also induces increase in free fatty acid levels, abnormal secretion of adipocytokine, increase in insulin resistance, and increase in blood sugar levels, i.e. serum glucose levels, resulting in development of diabetes.

Furthermore, increase in insulin resistance induces hyperinsulinemia, which leads to the development of hypertension. The increase in insulin resistance also induces hypertriglyceridemia and hypoalphalipoproteinemia, which lead to the development of hyperlipidemia. Accumulation of visceral fat caused by excessive calorie intake also induces increase in free fatty acid levels and increase in synthesis of liver triglycerides, resulting in the development of fatty liver.

Furthermore, the aforementioned increase in synthesis of liver triglycerides also induces increase in synthesis and secretion of VLDL, increase in VLDL, as well as generation and increase of free fatty acid, all of which lead to accumulation of visceral fat, resulting in obesity. Other possible causes of metabolic syndrome include increase in LDL levels, hyperlipidemia, and increase in VLDL remnants, which is caused by increase in VLDL. Furthermore, having metabolic syndrome synergistically increases risk of atherosclerosis.

What is claimed is:

1. A composition for controlling disorders induced by excessive calorie intake, said composition containing, at least, Hercampuri and having a function of controlling an interlinked complex of diseases induced by excessive calorie intake.

2. A composition as claimed in claim 1, wherein:
said composition has a function of controlling lifestyle-related disorders induced by accumulation of visceral fat.

3. A composition as claimed in claim 1, wherein:
said composition has a function of controlling diabetes.

4. A composition as claimed in claim 1, wherein:
said composition has a function of controlling Type 2 diabetes.

5. A composition as claimed in claim 1, wherein:
said composition has a function of improving serum lipid conditions and liver lipid conditions.

6. A composition as claimed in claim 1, wherein:
said composition has a function of lowering serum glucose levels.

7. A composition as claimed in claim 1, wherein:
said composition has a function of improving free fatty acid levels.

8. A composition as claimed in claim 1, wherein:
said composition has a function of reducing insulin resistance.

9. A composition as claimed in claim 1, wherein:
said composition has a function of decreasing cholesterol levels.

10. A composition as claimed in claim 1, wherein:
said composition has a function of reducing fat.

11. A composition as claimed in claim 1, wherein:
said composition has a function of reducing visceral fat.

12. A composition as claimed in claim 1, wherein:
said composition has a function of reducing subcutaneous fat.

13. A composition as claimed in claim 1, wherein:
said composition has preventing and curative properties effective against risk factors for metabolic syndrome.

14. A composition as claimed in claim 1, wherein:
said composition contains, at least, alcohol-extracted Hercampuri.

15. A composition as claimed in claim 1, wherein:
said composition contains, at least, Hercampuri extracted with aqueous alcohol containing 60 to 80% ethanol.

16. A food product for controlling disorders induced by excessive calorie intake, said food product containing a composition for controlling disorders induced by excessive calorie intake claimed in claim 1.

17. A skin preparation for external use for controlling disorders induced by excessive calorie intake, said skin preparation containing a composition for controlling disorders induced by excessive calorie intake claimed in claim 1.

18. A controlling agent for controlling disorders induced by excessive calorie intake, said controlling agent containing a composition for controlling disorders induced by excessive calorie intake claimed in claim 1.

19. A composition as claimed in claim 1, wherein:
Hercampuri is in a form of powder, granule, tablet, sugar-coated tablet, capsule, liquid, or syrup.

20. A composition as claimed in claim 19, wherein:
said composition further includes gelatin, various saccharides, starch, fatty acids, their salts, fats and oils, talc, physiological saline, or other masking agents.

21. A method of controlling diabetes, comprising:
administering to a subject a composition containing a range of 0.01 g to 10 g of Hercampuri.

22. A method as claimed in claim 21, wherein:
Hercampuri is in a form of powder, granule, tablet, sugar-coated tablet, capsule, liquid, or syrup.

23. A method as claimed in claim 22, wherein:
said composition further includes gelatin, various saccharides, starch, fatty acids, their salts, fats and oils, talc, physiological saline, or other masking agents.
